(19) United States
(12) Patent Application Publication
(10) Pub. No.: US 2018/0181716 A1
(43) Pub. Date: Jun. 28, 2018

(54) ROLE-BASED NAVIGATION INTERFACE SYSTEMS AND METHODS

(71) Applicant: General Electric Company, Schenectady, NY (US)

(72) Inventors: Amanda Ropa Mander, Seattle, WA (US); Hilari Scott, Baxter, TN (US)

(21) Appl. No.: 15/456,156

(22) Filed: Mar. 10, 2017

Related U.S. Application Data
(63) Continuation-in-part of application No. 15/391,513, filed on Dec. 27, 2016.

(51) Int. Cl. G06F 19/00; G06F 17/30; G06Q 10/06

(52) U.S. Cl. CPC G06F 19/322; G06F 3/0483; G06Q 10/0633; G06F 17/30979

(57) ABSTRACT

Certain examples provide role-based navigation interface systems and methods. An example apparatus includes a rules engine including rules for workflow execution and interface generation. The example apparatus includes an interface engine to generate a graphical user interface based on a role of a user and one or more rules. The interface engine is to at least: query the rules engine to provide the one or more rules based on the role; retrieve content based on the role and the one or more rules; dynamically generate the graphical user interface showing a first view based on the retrieved content arranged according to the role and the one or more rules; facilitate execution of a healthcare workflow by the user via the graphical user interface; and toggle between the first view and a second view, the second view dynamically generated based on the retrieved content arranged according to the role and the one or more rules.

### CROSS-REFERENCE TO RELATED APPLICATIONS

This patent arises from a continuation-in-part of U.S. patent application Ser. No. 15/391,513, entitled “System and Methods for Patient-Provider Engagement”, filed Dec. 27, 2016, which is incorporated herein by reference in its entirety.

### BACKGROUND

The statements in this section merely provide background information related to the disclosure and may not constitute prior art.

A challenge of current Electronic Medical Record (EMR) systems is a limited ability to support the complexity of medical practice activities. Activities include management, registration, rooming, encounter, visit summary, and billing. Current EMRs are limited to the encounter activity, large on-premise solutions or bolt-ons to existing systems add value or close technology gaps. Many EMRs were built when the technology was not as robust, and data entry was more valuable than data aggregation and big data management.

Additionally, with new regulations forcing a pay for performance healthcare system, patient compliance and evidence of care becomes increasingly important. Currently, non-discreet data is entered into the patient chart as a care plan, but that data is not searchable or analyzable as non-discreet data. Providers have a limited amount of time and cognitive energy and cannot devote time to properly understand the available data. Patients are unable to view and interact with their information appropriately to facilitate care compliance.

### BRIEF SUMMARY

Certain examples provide role-based navigation interface systems and methods.

An example apparatus includes a rules engine including rules for workflow execution and interface generation. The example apparatus includes an interface engine to generate a graphical user interface based on a role of a user and one or more rules. The interface engine is to at least: query the rules engine to provide the one or more rules based on the role; retrieve content based on the role and the one or more rules; dynamically generate the graphical user interface showing a first view based on the retrieved content arranged according to the role and the one or more rules; facilitate execution of a healthcare workflow by the user via the graphical user interface; and toggle between the first view and a second view, the second view dynamically generated based on the retrieved content arranged according to the role and the one or more rules.

An example computer-implemented method includes identifying, by executing an instruction using a processor, a role associated with a user. The example method includes querying, by executing an instruction using the processor, a rules engine to provide one or more rules based on the role. The example method includes retrieving, by executing an instruction using the processor, content based on the role and the one or more rules. The example method includes dynamically generating, by executing an instruction using the processor, a graphical user interface showing a first view based on the retrieved content arranged according to the role and the one or more rules. The example method includes facilitating, by executing an instruction using the processor, execution of a healthcare workflow by the user via the graphical user interface. The example method includes toggling, by executing an instruction using the processor, between the first view and a second view, the second view dynamically generated based on the retrieved content arranged according to the role and the one or more rules.

An example tangible computer-readable storage medium includes instructions. The instructions, when executed, particularly configure a processor to at least: identify a role associated with a user; query a rules engine to provide one or more rules based on the role; retrieve content based on the role and the one or more rules; dynamically generate a graphical user interface showing a first view based on the retrieved content arranged according to the role and the one or more rules; facilitate execution of a healthcare workflow by the user via the graphical user interface; and toggle between the first view and a second view, the second view dynamically generated based on the retrieved content arranged according to the role and the one or more rules.

Example computer-readable media, systems, and/or other apparatus can be used to implement methods disclosed herein.

### BRIEF DESCRIPTION OF THE DRAWINGS

The features and technical aspects of the system and method disclosed herein will become apparent in the following Detailed Description set forth below when taken in conjunction with the drawings in which like reference numerals indicate identical or functionally similar elements.

FIG. 1 shows a block diagram of an example healthcare-focused information system.

FIG. 2 shows a block diagram of an example healthcare information infrastructure including one or more systems.

FIG. 3 shows an example industrial internet configuration including a plurality of health-focused systems.

FIG. 4 illustrates an example clinical decision support system including an electronic medical record system and a rules engine.

FIG. 5 illustrates an example system in which the electronic medical record works with a cloud database and a batch rule engine.

FIG. 6 illustrates an example rules engine application container.

FIG. 7 illustrates a flow diagram of an example rules engine execution flow among rules engine components to execute a rule for a given input.

FIG. 8 illustrates a flow diagram of an example method for a batch process flow to provide recommendation and/or other data to a clinician based on patient history and facts.

FIG. 9 shows an example rules engine implemented in a cloud and interacting with a local, on-premise information system.

FIG. 10 illustrates an example clinical decision support system including a data and rules repository.

FIG. 11 illustrates an example implementation of a rules engine and associated repository embedded in one or more other clinical applications.

FIG. 12 provides a data flow for clinical quality reporting using the rules engine.

FIG. 13 illustrates an architectural block diagram of an example clinical decision support system including the rules engine.

FIG. 14 illustrates another example block diagram of a system integrating a partner system with a clinical decision support system and a clinical practice solutions and/or electronic medical records system.

FIG. 15 illustrates an example clinical decision support system leveraging the rules engine as part of the clinical decision support to generate an improved patient outcome.

FIG. 16 illustrates an example system implementing the rules engine and clinical decision support in the form of a workflow processor, a phase monitor, a rules engine, an interface engine, and an electronic medical records system.

FIG. 17 illustrates a visualization of a journey map for a healthcare workflow for a patient.

FIG. 18 illustrates a flow diagram of an example method of patient care plan composition, monitoring, and adjustment using the example workflow processor, phase monitor, rules engine, and electronic medical records.

FIG. 19 illustrates an example user interface framework.

FIGS. 20-33 illustrate an example curated landing page and related graphical user interface views.

FIG. 34 illustrates an example implementation of the interface engine of FIG. 16.

FIG. 35 illustrates a flow diagram of an example method of user interface generation and healthcare workflow management.

FIG. 36 is a block diagram of an example processor platform capable of executing instructions to implement the example systems and methods of FIGS. 1-18.

### DETAILED DESCRIPTION

In the following detailed description, reference is made to the accompanying drawings that form a part hereof, and in which is shown by way of illustration specific examples that may be practiced. These examples are described in sufficient detail to enable one skilled in the art to practice the subject matter, and it is to be understood that other examples may be utilized and that logical, mechanical, electrical and other changes may be made without departing from the scope of the subject matter of this disclosure. The following detailed description is, therefore, provided to describe an exemplary implementation and not to be taken as limiting on the scope of the subject matter described in this disclosure. Certain features from different aspects of the following description may be combined to form yet new aspects of the subject matter discussed below.

When introducing elements of various embodiments of the present disclosure, the articles “a,” “an,” “the,” and “said” are intended to mean that there are one or more of the elements. The terms “comprising,” “including,” and “having” are intended to be inclusive and mean that there may be additional elements other than the listed elements.

### I. Overview

Engaging ambulatory healthcare providers is a cumbersome process for patients that can lead to lack of adoption of preventative or chronic care measures for patients. By focusing on the patient user focus when building practice management capabilities, certain examples described herein facilitate increased interaction with ambulatory and/or other healthcare practices as the patient's first place to engage in their healthcare (e.g., their diagnosis, treatment, care plan, etc.). In addition, providing an improved interface for patient access, interaction, and participation should increase adherence to care plans. Certain examples described herein provide a foundation for a patient as a user who will access views of practice schedule, registration, pre-appointment tasks, post-appointment tasks, financial responsibility options, etc.

Historically, Practice Management (PM) and Electronic Medical Record (EMR) systems have focused on needs and wants of practice operations—documenting care, filing of claims, and balancing accounts receivable (AR). By focusing on both practice and patient functionality in a dynamic, interactive interface driven by an underlying rules engine understanding roles and available options when generating and executing the interface, certain examples described herein facilitate improved practice to patient relationships and interaction.

Certain examples provide a PM interface and underlying rules engine with a focus that allows patients to be users of a cloud-based solution. The PM interface helps ensure that patients can easily walk through coordination of their care with an ability to schedule appointments, understand financial responsibility with eligibility and patient liability estimation, manage registration updates online, identify open treatment or health plans and take action, for example.

Certain examples address a lack of patient engagement in the healthcare system due to cumbersome manual process(es) and lack of synchronous communication between care providers and patients. Certain examples help to generate an overall understanding of the impact of a care plan and clinical support team on patient health and simplification of the financial process to enable patients to focus on their care rather than their medical bills.

In certain examples, patients can access their care information in an easy, collaborative and mobile way to facilitate increased patient adoption of care via care plans, etc., according to one or more rules from a rules engine. Certain examples aid in provider practice management while also engaging the patient for improved care.

Mobile access provides patient solutions that streamline workflows with exception-based tasking for practices and patients to engage in healthcare. Certain examples allow practices and patients to manage appointment scheduling, pre-appointment work items, financial responsibility, coordinating gaps in care adherence, automating claims, and payment, and additional electronic data interchange (EDI) transactions. An associated interface provides a navigation that curates views based on user role. At login, depending on permissions, the user sees content related to their role in the practice management system.

For example, a graphical user interface (GUI) provides a macro view across all patients and a micro view for detailed patient information. By switching between these views, the provider can easily work with information at two levels. Because of the provider role, certain information specifically valuable for the provider is displayed in each view. Similarly, the patient can view information according to his or her micro view.

Providers and other users of electronic health records (EHRs)/electronic medical records (EMRs) are always time restrained. Patients are less likely to pay attention if too much information is provided to them as well. By showing users only the information that they need in that moment, workflows can be streamlined and processes sped up to give users back some time.

Thus, smart technology is used to surface information on demand via the interface, which de-clutters the interface for users and enables them to perform their tasks more quickly. A smart, rule-based engine drives the interface, which can provide practice management, EMR/EHR, and/or other medical software functionality.

Care plans are discreet and/or non-discreet data entered into the electronic medical record organized by each care event. A care plan is organized by problem or condition and represents standard(s) and protocol(s) for patient care based on payer and clinical guideline(s). By organizing and correlating discrete and non-discrete care pathway data in a medical record to clinical problem(s), condition(s) and/or comorbidities, care plans become repeatable and measurable standards of care. Care plans can be based on a specific payer, clinical standards of practice, and/or clinician specific guidelines, for example. Furthermore, problem oriented organization of data relevant to the problem and/or condition becomes automatable and actionable at the point of care when relevant.

A care pathway uses EMR/EHR and payer data to recommend intervention(s), activities and/or tasks appropriate for a patient's problem or set of problems. Productivity can be further improved by using a rules engine in conjunction with data input automating workflow, activities and/or tasks, when possible, based on clinical decision support intervention and/or other types of rules. The rules drive automation for an EMR system using tasking, alerts, and/or recommendations, for example. Thus, various portions of medical practice activities (e.g., management, registration, rooming, encounter, visit summary, billing) can be completed by facilitating the workflow to eliminate manual processes. Care pathway recommendations and/or workflow elements that cannot be automated are served up in the system workflow for manual intervention in orchestration with the automated recommendations, workflow, activities and/or tasks, for example.

Certain examples use a role-based rules system to determine access, workflows, activities, tasks, alerts and suggestions to be presented to complete activities. The system also allows for both role-based rules and individual custom rules to be created. Certain examples provide an ability to drive page content navigation to complete tasks.

An example of care plans with care pathways organized by problem and/or condition is as follows. Suppose a patient is over the age of 13 and is consulting medical professional for problem and/or condition of routine medical care. Routine correlated rules advise a practitioner to inquire if the patient smokes. An inquiry response can be extrapolated into preventative actions and/or more complex problems and/or conditions related to smoking that should be considered in the care pathway for the patient. Furthermore, if the patient has family or other relevant histories documented, rules can advise interventions such as a mammogram for breast cancer screening.

Clinical goals allow for a provider to compare patient data to a reference data point as well as set a smaller achievable goal. For example, regardless of whether a patient value is out of a reference range, a patient may have a more achievable goal. A clinical goal allows a provider to provide evidence-based care and tracking of patient compliance. Certain examples provide system and methods to relate reference values to a patient result and allow for a provider to set other patient-specific goals.

Certain examples provide systems and methods to facilitate clinical problem and guideline-based clinical care through problem-oriented organization of clinical information, care plans, pathways, and goals. An example workflow introduces clinical information organized and correlated by medical decision (problem) in conjunction with guidelines and decision support at the point of care and assists users (e.g., EMR users, clinical practice management/clinical practice solution system (CPS) users, etc.) to identify gaps in patient care and improve care quality through structured care plans inside the workflow. In certain examples, productivity can be further improved by automating workflow based on clinical decision support and business process rules.

With new regulations forcing a pay-for-performance healthcare system, patient compliance and evidence of care becomes increasingly important. Providers have a limited amount of time and cognitive energy to apply to patient care. By providing a solution that decreases the amount of cognitive load and time required for initial and mundane diagnostics, providers can instead exert their cognitive energy on more complex conditions.

Organizing structured care plans, pathway, and goals around problems and/or conditions can help EMR users decrease cognitive load while inside their workflow to identify gaps in patient care and opportunities to improve care quality. Benchmark goals can help to prevent and improve overall health. By intervening earlier patient outcomes are also improved, medical cost of a life time decreases. Medical evidence is also validated or improved upon.

In prior systems, non-discrete data is entered into a patient chart as one or more care plans. In certain examples, by correlating the non-discrete information to a clinical condition and comorbidities, problem-oriented views of clinical data can be generated that are actionable at a point of care. Furthermore, parsing out clinical goals transforms the non-discrete data into discrete data that is searchable. Certain examples allow additional rules to be run and become part of decision making at a point of care to enable clinicians to act on the data.

Certain examples organize structured care plans, pathways and goals around problems and/or conditions to assist EMR and/or CPS users to identify gaps in patient care and improve care quality while inside their workflow. User productivity can be further improved by automating workflow based on clinical decision support and business process rules that provide standardization and reduce laborious and costly manual processes.

Organizations can create their own standards of care and/or protocols to be used as guidelines, processes and/or policies for the organizations. In certain examples, a plug-in product that authors guideline content that can be referenced outside of a patient visit workflow. By organizing by problems and conditions and tying them to rules and a rules engine, certain examples facilitate a workflow not achieved by prior EMR or CPS systems.

Alternatively, a plug-in product can be provided to author guideline content that can be referenced outside of a patient visit workflow, for example. Using machine learning technologies, unlabeled data can be provided around a subject area, such as smoking, and allow the system to determine points of intervention.

Prior EMRs are very transactional and involve much user data entry. Certain examples move the data entry burden and introduce some machine intelligence. Certain examples use system intelligence and power of data to improve user and patient workflow and patient treatment. For example, a patient is treated by listening, coming to a decision, and setting goals to evaluate how the patient is doing with the treatment. For example, if a user prescribes immoxicilin for 14 days, certain examples provide a listener device in the system to process the order amount and time. The listener identifies when the patient has picked up the prescription from a pharmacy, and, if the listener does not detect a transaction to indicate that the patient has picked up his or her prescription, then the listener device triggers a task/activity to contact patient and inquire regarding the prescription, contact the pharmacy, etc., to help ensure there is no barrier to patient pick up of the prescription (e.g., patient cannot afford a co-pay, patient has no transport to get to pharmacy, etc.). Thus, listeners can be wrapped around transactions and/or other background tasks in a patient care plan/workflow.

In certain examples, care plan goals can be associated with listener devices to monitor progress toward and/or achievement of those goals. For example, if a patient has problems related to low-density lipoprotein (LDL), a doctor can prescribe medication as well as recommend a modification to diet and exercise with a goal to reduce weight by three pounds in four weeks. A listener can be associated with the care plan and goal. If the listener follows and evaluates program and determines whether or not the goal has not been met in four weeks. Based on the outcome, recommendations can be generated (e.g., more intrusive interventions (e.g., weight loss counseling, hypnotherapist, medication, etc.) or less intrusive inventions, etc., based on positive or negative outcome).

Certain examples also automate patient care workflows. For example, if a physician places an order or refers a patient for an x-ray imaging, heart monitoring, etc., and the patient does not comply (e.g., because the patient does not have enough time, the patient does not have enough money, the patient is not interested, etc.), the physician is penalized for lack of care. A notification or alert regarding the penalty can be surfaced in the workflow such that the system identifies the order or referral as well as the lack of completion or follow-through and triggers a notification (e.g., a note to a care manager to follow up with the patient, etc.).

Certain examples also provide business process rules to indicate and/or document aspects of patient care to help ensure providers are paid correctly for work being done. A rule defines a timing and/or other condition for a corresponding behavior and can also include other behavior(s) to be executed in order to be paid by an insurer. In certain examples, the system parses and understands care protocol and payment guidelines and guides and guides a user through the guidelines to help ensure proper care and payment.

In certain examples, ambulatory data (e.g., some discrete data and some non-discrete data, etc.) is stored in a data repository, such as an EMR, and an authoring tool is provided to author medical guidelines in real time and/or substantially real time (e.g., given data transmission, processing, storage, and/or retrieval delay) with clinical decision support. For example, a user can author rules that indicate if certain conditions are met, then corresponding activities are executed. For example, if a diabetes code is identified and the patient is over age 45, then the patient is recommended for a foot examination every year. Rules can be written to leverage the environment and terminology.

For example, suppose a patient is a diabetic and should get an A1C every year. A terminology service looks for a Logical Observation Identifiers Names and Codes (LOINC) code of A1C, which triggers a rule to search for an A1C appointment on the patient's chart. If such an appointment is not found, the rule suggests and/or makes an A1C appointment for the patient to evaluate that patient's glycated hemoglobin A1C level of average (e.g., 3 month) plasma glucose concentration.

In another example, the system can evaluate EMR ambulatory data and use a rules engine to apply rules and produce a recommendation. The recommendation can be displayed via a graphical user interface (GUI), and a user can then order a hemoglobin A1C for the patient. Once the A1C result comes back and does not satisfy a parameter within a normal range, an additional recommendation can indicate more frequent A1C reviews, increase medication, etc. In certain examples, a payer can have a similar rule to the provider A1C rule recommending an A1C review every three months. The payer rule can come into the repository and trump the provider rule of once a year to set the schedule more frequently and bridge the information loop, for example.

In certain examples, data, rules, and/or processing, etc., can be located on-premises at a healthcare facility. In certain examples, data, rules, and/or processing, etc., can be provided in a cloud-based implementation (e.g., run through a cloud storage factory with recommendations manifesting in cloud orders, etc.). In a cloud-based implementation, an on-premise database is located in a cloud, so customers do not have to migrate data to a different database. Instead, users have a common data factory to leverage that data for batch rules, clinical decision support, etc.

Thus, certain examples facilitate improved care quality through application of rules, monitoring, and improved patient outcomes (e.g., patients are cheaper to care for, less likely to have certain events, etc.). Certain examples provide actionable insights at a right time and place. Information is provided inside a workflow at a point at which the information is relevant to the workflow. A rule executes and a result appears inside an order screen where a user is placing the order, for example. A recommendation modifies a user's screen in real time to impact his or her workflow in the moment when/where the user is making decisions related to the recommendation.

As will be described further below, certain examples can integrate with and operate in a variety of healthcare environments and impact a variety of healthcare scenarios and data through sensing, decision support, workflow management, and control. The following section provides some context and example environment for the presently disclosed technology described further in the subsequent section below.

### II. Example Operating Environments

Health information, also referred to as healthcare information and/or healthcare data, relates to information generated and/or used by a healthcare entity. Health information can be information associated with health of one or more patients, for example. Health information may include protected health information (PHI), as outlined in the Health Insurance Portability and Accountability Act (HIPAA), which is identifiable as associated with a particular patient and is protected from unauthorized disclosure. Health information can be organized as internal information and external information. Internal information includes patient encounter information (e.g., patient-specific data, aggregate data, comparative data, etc.) and general healthcare operations information, etc. External information includes comparative data, expert and/or knowledge-based data, etc. Information can have both a clinical (e.g., diagnosis, treatment, prevention, etc.) and administrative (e.g., scheduling, billing, management, etc.) purpose.

Institutions, such as healthcare institutions, having complex network support environments and sometimes chaotically driven process flows utilize secure handling and safeguarding of the flow of sensitive information (e.g., personal privacy). A need for secure handling and safeguarding of information increases as a demand for flexibility, volume, and speed of exchange of such information grows. For example, healthcare institutions provide enhanced control and safeguarding of the exchange and storage of sensitive patient PHI and employee information between diverse locations to improve hospital operational efficiency in an operational environment typically having a chaotic-driven demand by patients for hospital services. In certain examples, patient identifying information can be masked or even stripped from certain data depending upon where the data is stored and who has access to that data. In some examples, PHI that has been “de-identified” can be re-identified based on a key and/or other encoder/decoder.

A healthcare information technology infrastructure can be adapted to service multiple business interests while providing clinical information and services. Such an infrastructure may include a centralized capability including, for example, a data repository, reporting, discrete data exchange/connectivity, “smart” algorithms, personalization/consumer decision support, etc. This centralized capability provides information and functionality to a plurality of users including medical devices, electronic records, access portals, pay for performance (P4P), chronic disease models, and clinical health information exchange/regional health information organization (HIE/RHIO), and/or enterprise pharmaceutical studies, home health, for example.

Interconnection of multiple data sources helps enable an engagement of all relevant members of a patient's care team and helps improve an administrative and management burden on the patient for managing his or her care. Particularly, interconnecting the patient's electronic medical record and/or other medical data can help improve patient care and management of patient information. Furthermore, patient care compliance is facilitated by providing tools that automatically adapt to the specific and changing health conditions of the patient and provide comprehensive education and compliance tools to drive positive health outcomes.

In certain examples, healthcare information can be distributed among multiple applications using a variety of database and storage technologies and data formats. To provide a common interface and access to data residing across these applications, a connectivity framework (CF) can be provided which leverages common data and service models (CDM and CSM) and service oriented technologies, such as an enterprise service bus (ESB) to provide access to the data.

In certain examples, a variety of user interface frameworks and technologies can be used to build applications for health information systems including, but not limited to, MICROSOFT® ASP.NET, AJAX®, MICROSOFT® Windows Presentation Foundation, GOOGLE® Web Toolkit, MICROSOFT® Silverlight, ADOBE®, and others. Applications can be composed from libraries of information widgets to display multi-content and multi-media information, for example. In addition, the framework enables users to tailor layout of applications and interact with underlying data.

In certain examples, an advanced Service-Oriented Architecture (SOA) with a modern technology stack helps provide robust interoperability, reliability, and performance. Example SOA includes a three-fold interoperability strategy including a central repository (e.g., a central repository built from Health Level Seven (HL7) transactions), services for working in federated environments, and visual integration with third-party applications. Certain examples provide portable content enabling plug 'n play content exchange among healthcare organizations. A standardized vocabulary using common standards (e.g., LOINC, SNOMED CT, RxNorm, FDB, ICD-9, ICD-10, etc.) is used for interoperability, for example. Certain examples provide an intuitive user interface to help minimize end-user training. Certain examples facilitate user-initiated launching of third-party applications directly from a desktop interface to help provide a seamless workflow by sharing user, patient, and/or other contexts. Certain examples provide real-time (or at least substantially real time assuming some system delay) patient data from one or more information technology (IT) systems and facilitate comparison(s) against evidence-based best practices. Certain examples provide one or more dashboards for specific sets of patients. Dashboard(s) can be based on condition, role, and/or other criteria to indicate variation(s) from a desired practice, for example.

A. Example Healthcare Information System

An information system can be defined as an arrangement of information/data, processes, and information technology that interact to collect, process, store, and provide informational output to support delivery of healthcare to one or more patients. Information technology includes computer technology (e.g., hardware and software) along with data and telecommunications technology (e.g., data, image, and/or voice network, etc.).

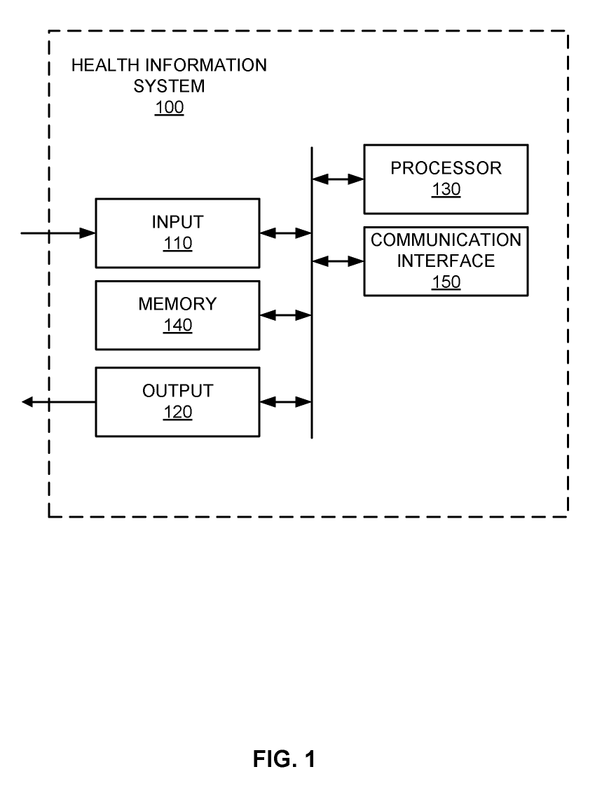

Turning now to the figures, FIG. 1 shows a block diagram of an example healthcare-focused information system 100. Example system 100 can be configured to implement a variety of systems and processes including image storage (e.g., picture archiving and communication system (PACS), etc.), image processing and/or analysis, radiology reporting and/or review (e.g., radiology information system (RIS), etc.), computerized provider order entry (CPOE) system, clinical decision support, patient monitoring, population health management (e.g., population health management system (PHMS), health information exchange (HIE), etc.), healthcare data analytics, cloud-based image sharing, electronic medical record (e.g., electronic medical record system (EMR), electronic health record system (EHR), electronic patient record (EPR), personal health record system (PHR), etc.), and/or other health information system (e.g., clinical information system (CIS), hospital information system (HIS), patient data management system (PDMS), laboratory information system (LIS), cardiovascular information system (CVIS), etc.

As illustrated in FIG. 1, the example information system 100 includes an input 110, an output 120, a processor 130, a memory 140, and a communication interface 150. The components of example system 100 can be integrated in one device or distributed over two or more devices.

Example input 110 may include a keyboard, a touch-screen, a mouse, a trackball, a track pad, optical barcode recognition, voice command, etc. or combination thereof used to communicate an instruction or data to system 100. Example input 110 may include an interface between systems, between user(s) and system 100, etc.

Example output 120 can provide a display generated by processor 130 for visual illustration on a monitor or the like. The display can be in the form of a network interface or graphic user interface (GUI) to exchange data, instructions, or illustrations on a computing device via communication interface 150, for example. Example output 120 may include a monitor (e.g., liquid crystal display (LCD), plasma display, cathode ray tube (CRT), etc.), light emitting diodes (LEDs), a touch-screen, a printer, a speaker, or other conventional display device or combination thereof.

Example processor 130 includes hardware and/or software configuring the hardware to execute one or more tasks and/or implement a particular system configuration. Example processor 130 processes data received at input 110 and generates a result that can be provided to one or more of output 120, memory 140, and communication interface 150. For example, example processor 130 can take user annotation provided via input 110 with respect to an image displayed via output 120 and can generate a report associated with the image based on the annotation. As another example, processor 130 can process updated patient information obtained via input 110 to provide an updated patient record to an EMR via communication interface 150.

Example memory 140 may include a relational database, an object-oriented database, a data dictionary, a clinical data repository, a data warehouse, a data mart, a vendor neutral archive, an enterprise archive, etc. Example memory 140 stores images, patient data, best practices, clinical knowledge, analytics, reports, etc. Example memory 140 can store data and/or instructions for access by the processor 130. In certain examples, memory 140 can be accessible by an external system via the communication interface 150.

In certain examples, memory 140 stores and controls access to encrypted information, such as patient records, encrypted update-transactions for patient medical records, including usage history, etc. In an example, medical records can be stored without using logic structures specific to medical records. In such a manner, memory 140 is not searchable. For example, a patient's data can be encrypted with a unique patient-owned key at the source of the data. The data is then uploaded to memory 140. Memory 140 does not process or store unencrypted data thus minimizing privacy concerns. The patient's data can be downloaded and decrypted locally with the encryption key.

For example, memory 140 can be structured according to provider, patient, patient/provider association, and document. Provider information may include, for example, an identifier, a name, and address, a public key, and one or more security categories. Patient information may include, for example, an identifier, a password hash, and an encrypted email address. Patient/provider association information may include a provider identifier, a patient identifier, an encrypted key, and one or more override security categories. Document information may include an identifier, a patient identifier, a clinic identifier, a security category, and encrypted data, for example.

Example communication interface 150 facilitates transmission of electronic data within and/or among one or more systems. Communication via communication interface 150 can be implemented using one or more protocols. In some examples, communication via communication interface 150 occurs according to one or more standards (e.g., Digital Imaging and Communications in Medicine (DICOM), Health Level Seven (HL7), ANSI X12N, etc.). Example communication interface 150 can be a wired interface (e.g., a data bus, a Universal Serial Bus (USB) connection, etc.) and/or a wireless interface (e.g., radio frequency, infrared (IR), near field communication (NFC), etc.). For example, communication interface 150 may communicate via wired local area network (LAN), wireless LAN, wide area network (WAN), etc. using any past, present, or future communication protocol (e.g., BLUETOOTH™, USB 2.0, USB 3.0, etc.).

In certain examples, a Web-based portal may be used to facilitate access to information, patient care and/or practice management, etc. Information and/or functionality available via the Web-based portal may include one or more of order entry, laboratory test results review system, patient information, clinical decision support, medication management, scheduling, electronic mail and/or messaging, medical resources, etc. In certain examples, a browser-based interface can serve as a zero footprint, zero download, and/or other universal viewer for a client device.

In certain examples, the Web-based portal serves as a central interface to access information and applications, for example. Data may be viewed through the Web-based portal or viewer, for example. Additionally, data may be manipulated and propagated using the Web-based portal, for example. Data may be generated, modified, stored and/or used and then communicated to another application or system to be modified, stored and/or used, for example, via the Web-based portal, for example.

The Web-based portal may be accessible locally (e.g., in an office) and/or remotely (e.g., via the Internet and/or other private network or connection), for example. The Web-based portal may be configured to help or guide a user in accessing data and/or functions to facilitate patient care and practice management, for example. In certain examples, the Web-based portal may be configured according to certain rules, preferences and/or functions, for example. For example, a user may customize the Web portal according to particular desires, preferences and/or requirements.

B. Example Healthcare Infrastructure

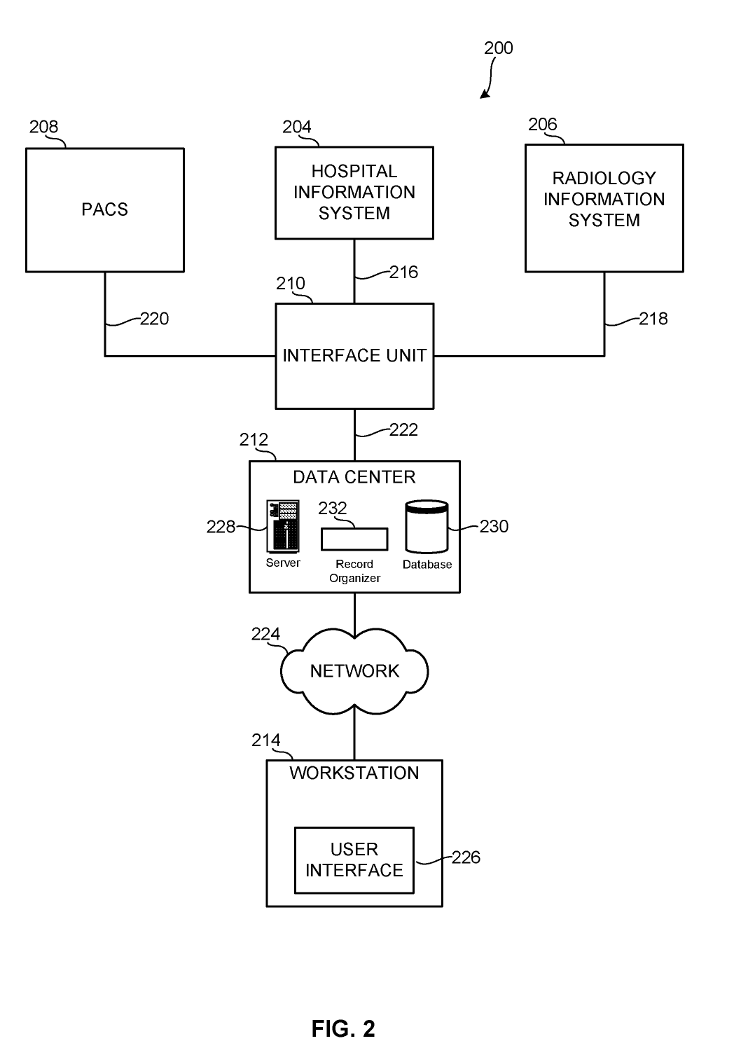

FIG. 2 shows a block diagram of an example healthcare information infrastructure 200 including one or more subsystems such as the example healthcare-related information system 100 illustrated in FIG. 1. Example healthcare system 200 includes a HIS 204, a RIS 206, a PACS 208, an interface unit 210, a data center 212, and a workstation 214. In the illustrated example, HIS 204, RIS 206, and PACS 208 are particular implementations of the healthcare-related information system 100 and are housed in a healthcare facility and locally archived. However, in other implementations, HIS 204, RIS 206, and/or PACS 208 may be housed within one or more other suitable locations. In certain implementations, one or more of PACS 208, RIS 206, HIS 204, etc., may be implemented remotely via a thin client and/or downloadable software solution. Furthermore, one or more components of the healthcare system 200 can be combined and/or implemented together. For example, RIS 206 and/or PACS 208 can be integrated with HIS 204; PACS 208 can be integrated with RIS 206; and/or the three example information systems 204, 206, and/or 208 can be integrated together. In other example implementations, healthcare system 200 includes a subset of the illustrated information systems 204, 206, and/or 208. For example, healthcare system 200 may include only one or two of HIS 204, RIS 206, and/or PACS 208. Information (e.g., scheduling, test results, exam image data, observations, diagnosis, etc.) can be entered into HIS 204, RIS 206, and/or PACS 208 by healthcare practitioners (e.g., radiologists, physicians, and/or technicians) and/or administrators before and/or after patient examination. One or more of the HIS 204, RIS 206, and/or PACS 208 can communicate with equipment and system(s) in an operating room, patient room, etc., to track activity, correlate information, generate reports and/or next actions, and the like.

The HIS 204 stores medical information such as clinical reports, patient information, and/or administrative information received from, for example, personnel at a hospital, clinic, and/or a physician's office (e.g., an EMR, EHR, PHR, etc.). RIS 206 stores information such as radiology reports, radiology exam image data, messages, warnings, alerts, patient scheduling information, patient demographic data, patient tracking information, and/or physician and patient status monitors. Additionally, RIS 206 enables exam order entry (e.g., ordering an x-ray of a patient) and image and film tracking (e.g., tracking identities of one or more people that have checked out a film). In some examples, information in RIS 206 is formatted according to the HL-7 (Health Level Seven) clinical communication protocol. In certain examples, a medical exam distributor is located in RIS 206 to facilitate distribution of radiology exams to a radiologist workload for review and management of the exam distribution by, for example, an administrator.

PACS 208 stores medical images (e.g., x-rays, scans, three-dimensional renderings, etc.) as, for example, digital images in a database or registry. In some examples, the medical images are stored in PACS 208 using the Digital Imaging and Communications in Medicine (DICOM) format. Images are stored in PACS 208 by healthcare practitioners (e.g., imaging technicians, physicians, radiologists) after a medical imaging of a patient and/or are automatically transmitted from medical imaging devices to PACS 208 for storage. In some examples, PACS 208 can also include a display device and/or viewing workstation to enable a healthcare practitioner or provider to communicate with PACS 208.

The interface unit 210 includes a hospital information system interface connection 216, a radiology information system interface connection 218, a PACS interface connection 220, and a data center interface connection 222. Interface unit 210 facilities communication among HIS 204, RIS 206, PACS 208, and/or data center 212. Interface connections 216, 218, 220, and 222 can be implemented by, for example, a Wide Area Network (WAN) such as a private network or the Internet. Accordingly, interface unit 210 includes one or more communication components such as, for example, an Ethernet device, an asynchronous transfer mode (ATM) device, an 802.11 device, a DSL modem, a cable modem, a cellular modem, etc. In turn, the data center 212 communicates with workstation 214, via a network 224, implemented at a plurality of locations (e.g., a hospital, clinic, doctor's office, other medical office, or terminal, etc.). Network 224 is implemented by, for example, the Internet, an intranet, a private network, a wired or wireless Local Area Network, and/or a wired or wireless Wide Area Network. In some examples, interface unit 210 also includes a broker (e.g., a Mitra Imaging's PACS Broker) to allow medical information and medical images to be transmitted together and stored together.

Interface unit 210 receives images, medical reports, administrative information, exam workload distribution information, and/or other clinical information from the information systems 204, 206, 208 via the interface connections 216, 218, 220. If necessary (e.g., when different formats of the received information are incompatible), interface unit 210 translates or reformats (e.g., into Structured Query Language (“SQL”) or standard text) the medical information, such as medical reports, to be properly stored at data center 212. The reformatted medical information can be transmitted using a transmission protocol to enable different medical information to share common identification elements, such as a patient name or social security number. Next, interface unit 210 transmits the medical information to data center 212 via data center interface connection 222. Finally, medical information is stored in data center 212 in, for example, the DICOM format, which enables medical images and corresponding medical information to be transmitted and stored together.

The medical information is later viewable and easily retrievable at workstation 214 (e.g., by their common identification element, such as a patient name or record number). Workstation 214 can be any equipment (e.g., a personal computer) capable of executing software that permits electronic data (e.g., medical reports) and/or electronic medical images (e.g., x-rays, ultrasounds, MRI scans, etc.) to be acquired, stored, or transmitted for viewing and operation. Workstation 214 receives commands and/or other input from a user via, for example, a keyboard, mouse, track ball, microphone, etc. Workstation 214 is capable of implementing a user interface 226 to enable a healthcare practitioner and/or administrator to interact with healthcare system 200. For example, in response to a request from a physician, user interface 226 presents a patient medical history. In other examples, a radiologist is able to retrieve and manage a workload of exams distributed for review to the radiologist via user interface 226. In further examples, an administrator reviews radiologist workloads, exam allocation, and/or operational statistics associated with the distribution of exams via user interface 226. In some examples, the administrator adjusts one or more settings or outcomes via user interface 226.

Example data center 212 of FIG. 2 is an archive to store information such as images, data, medical reports, and/or, more generally, patient medical records. In addition, data center 212 can also serve as a central conduit to information located at other sources such as, for example, local archives, hospital information systems/radiology information systems (e.g., HIS 204 and/or RIS 206), or medical imaging/storage systems (e.g., PACS 208 and/or connected imaging modalities). That is, the data center 212 can store links or indicators (e.g., identification numbers, patient names, or record numbers) to information. In the illustrated example, data center 212 is managed by an application server provider (ASP) and is located in a centralized location that can be accessed by a plurality of systems and facilities (e.g., hospitals, clinics, doctor's offices, other medical offices, and/or terminals). In some examples, data center 212 can be spatially distant from HIS 204, RIS 206, and/or PACS 208. The data center 212 can drive content for display and interaction via a graphical user interface, for example.

Example data center 212 of FIG. 2 includes a server 228, a database 230, and a record organizer 232. Server 228 receives, processes, and conveys information to and from the components of healthcare system 200. Database 230 stores the medical information described herein and provides access thereto. Example record organizer 232 of FIG. 2 manages patient medical histories, for example. Record organizer 232 can also assist in procedure scheduling, for example.

Certain examples can be implemented as cloud-based clinical information systems and associated methods of use. An example cloud-based clinical information system enables healthcare entities (e.g., patients, clinicians, sites, groups, communities, and/or other entities) to share information via web-based applications, cloud storage and cloud services. For example, the cloud-based clinical information system may enable a first clinician to securely upload information into the cloud-based clinical information system to allow a second clinician to view and/or download the information via a web application. Thus, for example, the first clinician may upload an x-ray image into the cloud-based clinical information system, and the second clinician may view the x-ray image via a web browser and/or download the x-ray image onto a local information system employed by the second clinician.

In certain examples, users (e.g., a patient and/or care provider) can access functionality provided by system 200 via a software-as-a-service (SaaS) implementation over a cloud or other computer network, for example. In certain examples, all or part of system 200 can also be provided via platform as a service (PaaS), infrastructure as a service (IaaS), etc. For example, system 200 can be implemented as a cloud-delivered Mobile Computing Integration Platform as a Service. A set of consumer-facing Web-based, mobile, and/or other applications enable users to interact with the PaaS, for example.

C. Industrial Internet Examples

The Internet of things (also referred to as the “Industrial Internet”) relates to an interconnection between a device that can use an Internet connection to talk with other devices on the network. Using the connection, devices can communicate to trigger events/actions (e.g., changing temperature, turning on/off, providing a status, etc.). In certain examples, machines can be merged with “big data” to improve efficiency and operations, provide improved data mining, facilitate better operation, etc.

Big data can refer to a collection of data so large and complex that it becomes difficult to process using traditional data processing tools/methods. Challenges associated with a large data set include data capture, sorting, storage, search, transfer, analysis, and visualization. A trend toward larger data sets is due at least in part to additional information derivable from analysis of a single large set of data, rather than analysis of a plurality of separate, smaller data sets. By analyzing a single large data set, correlations can be found in the data, and data quality can be evaluated.

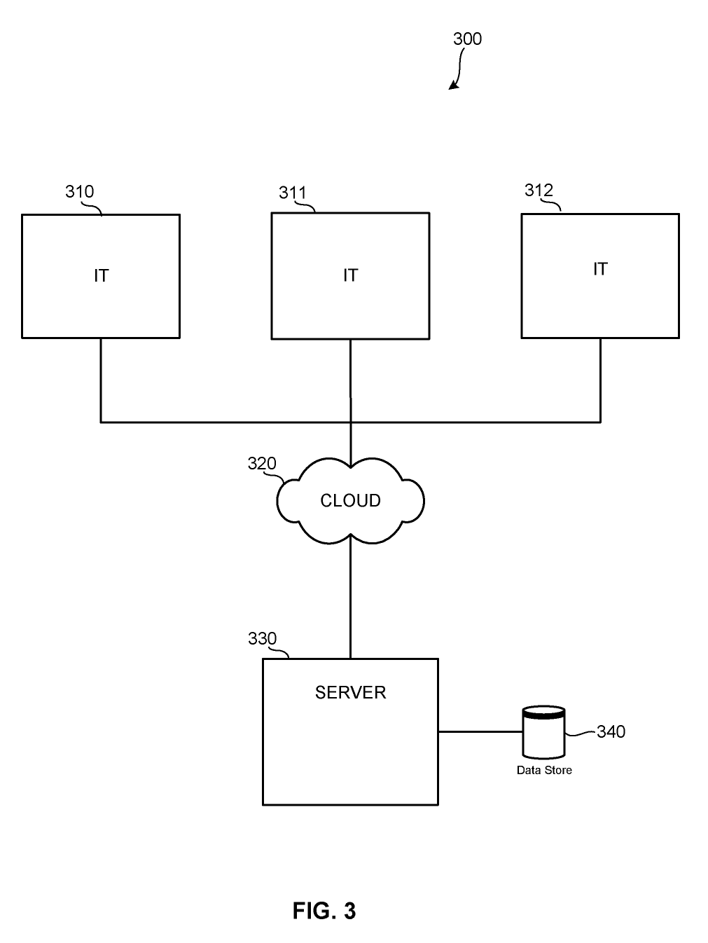

FIG. 3 illustrates an example industrial internet configuration 300. Example configuration 300 includes a plurality of health-focused systems 310-312, such as a plurality of health information systems 100 (e.g., PACS, RIS, EMR, etc.) communicating via industrial internet infrastructure 300. Example industrial internet 300 includes a plurality of health-related information systems 310-312 communicating via a cloud 320 with a server 330 and associated data store 340.

As shown in the example of FIG. 3, a plurality of devices (e.g., information systems, imaging modalities, etc.) 310-312 can access a cloud 320, which connects the devices 310-312 with a server 330 and associated data store 340. Information systems, for example, include communication interfaces to exchange information with server 330 and data store 340 via the cloud 320. Other devices, such as medical imaging scanners, patient monitors, etc., can be outfitted with sensors and communication interfaces to enable them to communicate with each other and with the server 330 via the cloud 320.

Thus, machines 310-312 within system 300 become “intelligent” as a network with advanced sensors, controls, analytical based decision support and hosting software applications. Using such an infrastructure, advanced analytics can be provided to associated data. The analytics combines physics-based analytics, predictive algorithms, automation, and deep domain expertise. Via cloud 320, devices 310-312 and associated people can be connected to support more intelligent design, operations, maintenance, and higher server quality and safety, for example.

Using the industrial internet infrastructure, for example, a proprietary machine data stream can be extracted from a device 310. Machine-based algorithms and data analysis are applied to the extracted data. Data visualization can be remote, centralized, etc. Data is then shared with authorized users, and any gathered and/or gleaned intelligence is fed back into the machines 310-312.

While progress with industrial equipment automation has been made over the last several decades, and assets have become ‘smarter,’ the intelligence of any individual asset pales in comparison to intelligence that can be gained when multiple smart devices are connected together. Aggregating data collected from or about multiple assets can enable users to improve business processes, for example by improving effectiveness of asset maintenance or improving operational performance if appropriate industrial-specific data collection and modeling technology is developed and applied.

In an example, an industrial asset can be outfitted with one or more sensors configured to monitor respective ones of an asset's operations or conditions. Data from the one or more sensors can be recorded or transmitted to a cloud-based or other remote computing environment. By bringing such data into a cloud-based computing environment, new software applications informed by industrial process, tools and know-how can be constructed, and new physics-based analytics specific to an industrial environment can be created. Insights gained through analysis of such data can lead to enhanced asset designs, or to enhanced software algorithms for operating the same or similar asset at its edge, that is, at the extremes of its expected or available operating conditions.

Systems and methods to manage industrial assets can include or can be a portion of an Industrial Internet of Things (IIoT). In an example, an IIoT connects industrial assets, such as turbines, jet engines, and locomotives, to the Internet or cloud, or to each other in some meaningful way. The systems and methods described herein can include using a “cloud” or remote or distributed computing resource or service. The cloud can be used to receive, relay, transmit, store, analyze, or otherwise process information for or about one or more industrial assets. In an example, a cloud computing system includes at least one processor circuit, at least one database, and a plurality of users or assets that are in data communication with the cloud computing system. The cloud computing system can further include or can be coupled with one or more other processor circuits or modules configured to perform a specific task, such as to perform tasks related to asset maintenance, analytics, data storage, security, or some other function.

However, the integration of industrial assets with the remote computing resources to enable the IIoT often presents technical challenges separate and distinct from the specific industry and from computer networks, generally. A given industrial asset may need to be configured with novel interfaces and communication protocols to send and receive data to and from distributed computing resources. Given industrial assets may have strict requirements for cost, weight, security, performance, signal interference, and the like such that enabling such an interface is rarely as simple as combining the industrial asset with a general purpose computing device.

To address these problems and other problems resulting from the intersection of certain industrial fields and the IIoT, embodiments may enable improved interfaces, techniques, protocols, and algorithms for facilitating communication with and configuration of industrial assets via remote computing platforms and frameworks. Improvements in this regard may relate to both improvements that address particular challenges related to particular industrial assets (e.g., improved imaging systems, medical records systems, analytics systems, etc.) that address particular problems related to use of these industrial assets with these remote computing platforms and frameworks, and also improvements that address challenges related to operation of the platform itself to provide improved mechanisms for configuration, analytics, and remote management of industrial assets.

The Predix™ platform available from GE is a novel embodiment of such Asset Management Platform (AMP) technology enabled by state of the art cutting edge tools and cloud computing techniques that enable incorporation of a manufacturer's asset knowledge with a set of development tools and best practices that enables asset users to bridge gaps between software and operations to enhance capabilities, foster innovation, and ultimately provide economic value. Through the use of such a system, a manufacturer of industrial assets can be uniquely situated to leverage its understanding of industrial assets themselves, models of such assets, and industrial operations or applications of such assets, to create new value for industrial customers through asset insights.

D. Data Mining Examples

Imaging informatics includes determining how to tag and index a large amount of data acquired in diagnostic imaging in a logical, structured, and machine-readable format. By structuring data logically, information can be discovered and utilized by algorithms that represent clinical pathways and decision support systems. Data mining can be used to help ensure patient safety, reduce disparity in treatment, provide clinical decision support, etc. Mining both structured and unstructured data from radiology reports, as well as actual image pixel data, can be used to tag and index both imaging reports and the associated images themselves.

E. Example Methods of Use

Clinical workflows are typically defined to include one or more steps or actions to be taken in response to one or more events and/or according to a schedule. Events may include receiving a healthcare message associated with one or more aspects of a clinical record, opening a record(s) for new patient(s), receiving a transferred patient, reviewing and reporting on an image, executing orders for specific care, signing off on orders for a discharge, and/or any other instance and/or situation that requires or dictates responsive action or processing. The actions or steps of a clinical workflow may include placing an order for one or more clinical tests, scheduling a procedure, requesting certain information to supplement a received healthcare record, retrieving additional information associated with a patient, providing instructions to a patient and/or a healthcare practitioner associated with the treatment of the patient, radiology image reading, dispatching room cleaning and/or patient transport, and/or any other action useful in processing healthcare information or causing critical path care activities to progress. The defined clinical workflows may include manual actions or steps to be taken by, for example, an administrator or practitioner, electronic actions or steps to be taken by a system or device, and/or a combination of manual and electronic action(s) or step(s). While one entity of a healthcare enterprise may define a clinical workflow for a certain event in a first manner, a second entity of the healthcare enterprise may define a clinical workflow of that event in a second, different manner. In other words, different healthcare entities may treat or respond to the same event or circumstance in different fashions. Differences in workflow approaches may arise from varying preferences, capabilities, requirements or obligations, standards, protocols, etc. among the different healthcare entities.

In certain examples, a medical exam conducted on a patient can involve review by a healthcare practitioner, such as a radiologist, to obtain, for example, diagnostic information from the exam. In a hospital setting, medical exams can be ordered for a plurality of patients, all of which require review by an examining practitioner. Each exam has associated attributes, such as a modality, a part of the human body under exam, and/or an exam priority level related to a patient criticality level. Hospital administrators, in managing distribution of exams for review by practitioners, can consider the exam attributes as well as staff availability, staff credentials, and/or institutional factors such as service level agreements and/or overhead costs.

Additional workflows can be facilitated such as bill processing, revenue cycle management, population health management, patient identity, consent management, etc. Such workflows, as well as the systems and workflows described above, are sufficiently complex that they cannot be implemented or executed manually by a human within a reasonable time period.

### III. Example Patient Navigation Systems and Methods

As described above, certain examples generate interfaces and provide access to information driven by user role and a rules engine to dynamically surface and/or hide information based on context, role, and relevance to streamline operation and facilitate care plan compliance for a patient and/or other user.

Certain examples provide an interface accessible via mobile (e.g., smartphone, tablet computer, etc.) and/or other computer interface (e.g., desktop computer, laptop computer, etc.). For example, mobile access provides patient solutions that streamline workflows with exception-based tasking for practices and patients to engage in healthcare. Certain examples allow practices and patients to manage appointment scheduling, pre-appointment work items, financial responsibility, coordinating gaps in care adherence, automating claims, and payment, and additional electronic data interchange (EDI) transactions. An associated interface provides a navigator that curates views based on user role. At login, depending on permissions, the user sees content related to his or her role in the practice management system.

For example, a graphical user interface (GUI) provides a macro view across all patients and a micro view for detailed patient information. By switching between these views, the provider can easily work with information at two levels. Because of the provider role, certain information specifically valuable for the provider is displayed in each view. Similarly, the patient can view information according to his or her micro view.

Providers and other users of electronic health records (EHRs)/electronic medical records (EMRs) are always time restrained. Patients are less likely to pay attention if too much information is provided to them as well. By showing users only the information that they need in that moment, workflows can be streamlined and processes sped up to give users back some time.

Thus, smart technology is used to surface information on demand via the interface, which de-clutters the interface for users and enables them to perform their tasks more quickly. A smart, rule-based engine drives the interface, which can provide practice management, EMR/EHR, and/or other medical software functionality.

In certain examples, rules determine available roles and available options, correlation options to roles, and dynamically render the GUI based on the correlation between options and roles. The GUI starting with a default for the role and dynamically rendering further views based on user selection and available options for that role. In certain examples, the configuration can remain cached to allow the user to flip between several interface views. A rules engine drives available options and dynamically alters the GUI based on selection and available information according to the rules.

Certain examples facilitate a patient journey and patient and/or provider workflow through rules engine-driven scenario mapping. Certain examples provide enhanced clinical decision support for task orders, suggestions, etc., as well as improved care management, case management, registration, billing, etc. Certain examples provide a care team ecosystem to facilitate a workflow for patient-provider interaction for patient treatment according to a care plan or pathway. Certain examples leverage rules and available data (e.g., electronic medical record data, device data, imaging data, financial data, resource status data, etc.) to drive tasks and react to failures in compliance to suggest provider action.

Certain examples leverage the patient-provider workflow and identify roles in a healthcare environment. The workflow is divided into phases, and, for each phase, rules are associated with each role. Each phase includes at least one activity specified by the rules, and each activity involves at least one role and at least one associated task, alert, and suggestion for the at least one role specified by the rules. In certain examples, the rules connect the activities of the phases of the workflow to an electronic medical record system to automatically provide and receive data for a patient in each phase of the workflow. The phases of the workflow along with activities and data form a patient journey map to guide the patient, healthcare providers, and healthcare systems through the workflow, for example. Feedback allows the system to provide intelligence for successes, failures, omissions, etc., to help ensure certain information (e.g., need a new copy of patient's insurance card, etc.) is available and used to help drive the workflow.

In a fee-for-service environment, patients can go to a variety of providers for service without penalty. Providers can enter into value-based contracts which give the provider responsibility for a certain number of patients (e.g., five thousand patients, etc.). A doctor can view his or her patients, track their activity, and track what they're being measured against.

Certain examples connecting payer and provider data in the workflow. Certain examples remove an intermediary and enable real-time feedback and authorization for procedures, orders, etc., by leveraging system APIs to communicate between disparate healthcare systems with a rules engine in between the systems. For example, if a physician requests a computed tomography (CT) scan for a patient, rule(s) provided by the rules engine indicate that such an order requires authorization because the patient has a certain insurance and a configuration file for that insurance indicates authorization is needed for the order, associated with a particular code (e.g., LOINC code, ICD-10 code, etc.). Certain examples surface this issue by alerting the provider through the connected system APIs and rules engine to prompt the provider to ask for authorization and confirm that they can/wish to proceed. If so, the systems contact the payer and obtain authorization to proceed. Then, when the order for a CT scan is transmitted to an imaging center, the authorization and other information/documentation is transmitted with the order so the imaging center does not need to follow up and ask for additional information, for example. The rules engine works with an electronic medical records system, billing system, and/or other healthcare system to facilitate the automated workflow between systems while reducing or minimizing human action, for example.

Certain examples provide a clinical decision support system 400 including an EMR 410 and a rules engine 420. The example EMR 410 can be operated in a real time and/or batch mode. In a real-time mode, the rule engine 420 is triggered at a plurality of preconfigured trigger points in the EMR application 410. The EMR 410 pulls clinical patient data (e.g., facts) from an EMR database and sends the patient data to the rule engine 420 to generate a recommendation. The rule engine 420 loads one or more rules and applies the one or more rules to the clinical facts to generate a recommendation. The EMR 410 stores the recommendation (e.g., in a database on the cloud, etc.) and outputs (e.g., displays, etc.) the recommendation to a clinician.

In certain examples, a batch mode is triggered when data is made available on the cloud. The batch process pulls clinical facts from the data on the cloud and applies clinical batch rules to generate a recommendation. The recommendation persists on the cloud at the end of the batch process. During a patient encounter, for example, when a clinician logs into the EMR 410, the recommendation is pulled and displayed to the user.

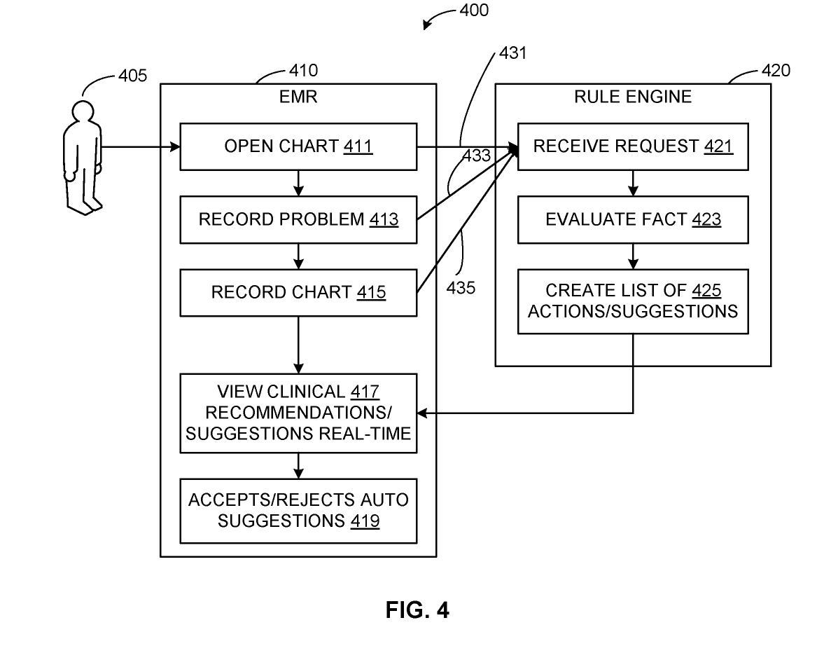

Turning to the example of FIG. 4, a patient 405 opens his or her patient chart 411 via the EMR 410. Opening the patient chart 411 triggers 431 the rule engine 420 to receive a request 421 for a recommendation. At the EMR 410, a patient problem is recorded 413, which also triggers 433 a request 421 at the rule engine 420. The patient chart is recorded 415 at the EMR 410, which triggers 435 another request 421 at the rule engine 420. The rule engine 420 evaluates facts 423 and creates a list of actions and/or suggestions 425 based on rules applied to the data. The rule engine 420 generates a response 437 based on the actions and/or suggestions. The EMR 410 receives the response 437 and, based on the response 437, generates clinical recommendations and/or suggestions 417 in real time and/or substantially real time given data retrieval, processing, and/or transmission delay. The EMR 410 accepts and/or rejects 419 the automatically generated recommendations and/or suggestions (e.g., based on user input, rules, other stored data, etc.).

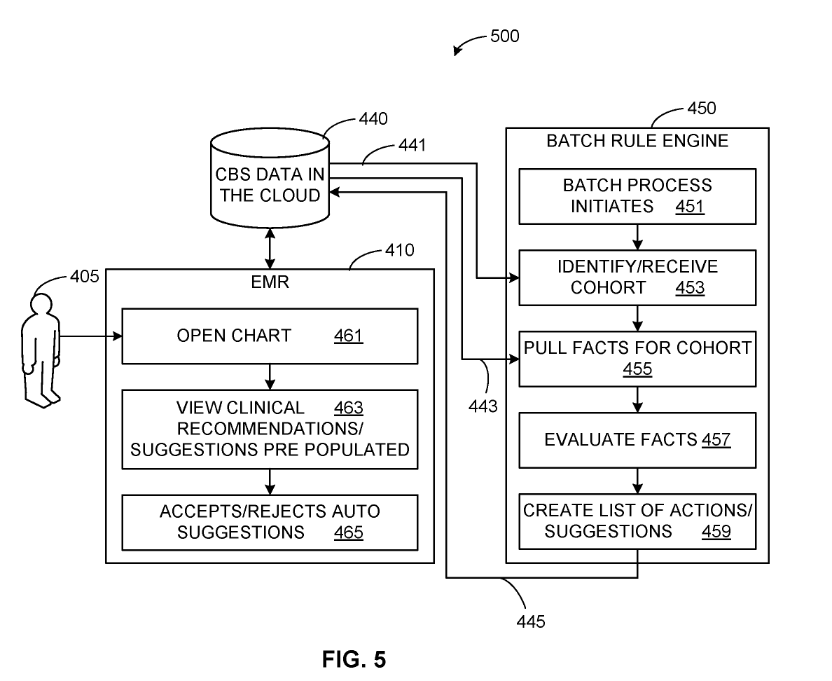

FIG. 5 illustrates an example system 500 in which the EMR 410 works with a cloud database 440 and a batch rule engine 450. As shown in the example of FIG. 5, the patient 405 opens his or her chart 461 via the EMR 410. Patient data is retrieved from and/or provided to the cloud database 440. The cloud-based data 440 is batched as a cohort 441 and provided to the batch rule engine 450. In the batch rule engine 450, a batch process initiates 451 and the cohort 441 is identified and/or received 453. The batch rule engine 450 pulls 455 facts 443 from the database 440 based on the cohort 441 and evaluates 457 the facts 443 with respect to rules of the batch rule engine 450. The batch rule engine 450 generates 459 a list of actions and/or suggestions provided as a response 445 to the cloud database 440 based on the actions and/or suggestions. The EMR 410 can query the cloud database 440 to view pre-populated clinical recommendations and/or suggestions 463 in real time and/or substantially real time given data retrieval, processing, and/or transmission delay. The EMR 410 accepts and/or rejects 465 the automatically generated recommendations and/or suggestions (e.g., based on user input, rules, other stored data, etc.).

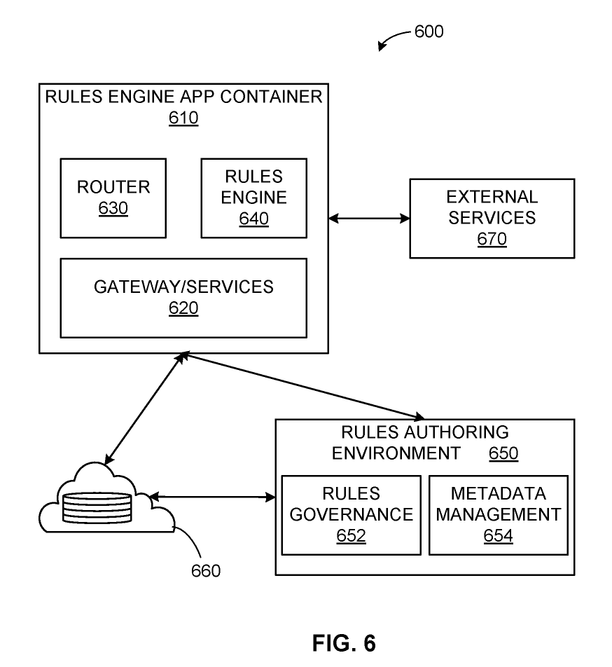

FIG. 6 illustrates an example rules engine application container 610 including a gateway/services 620, a router 630, and a rules engine 640 (e.g., the rules engine 420, rules engine 450, and/or other rules engine, etc.). The example gateway and/or other communication services 620 includes one or more end points, Representational State Transfer (REST) architecture with JavaScript Object Notation (JSON) for data interchange, discovery, audit, logging, security, monitoring, etc. The gateway services 620 provides a point of entry for rules engine 640 requests. An application program interface (API) for the rules engine 640 is exposed for access, such as via a REST endpoint over HTTPS, etc. An input and output payload can be implemented using a data model, such as a data model from the Fast Healthcare Interoperability Resources (FHIR) Draft Standard for Trial Use (DTSU 2) Specification. The gateway services 620 are responsible for cross-cutting functions such as logging, security, auditing, etc.

The example router 630 includes a rules set selector, rules metadata template(s), mapping, etc. The example rules engine 640 provides execution of rules according to a rule set/policy, for example. In certain examples, the router 630 fetches pre-requisite data for rule execution, validate clinical facts, and provide data and facts to the rules engine 640 to generate a recommendation. Once the rules engine 640 provides the recommendation, the router 630 post-processes the data before providing the data to the gateway server 620.

The example rules engine application container 610 interacts with a rules authoring environment 650. The example rules authoring environment 650 includes rules governance 652 and metadata management 654, for example. Rules can be stored in a storage 660, for example. The example rules engine application container 610 also interacts with one or more external services 670 such as a terminology service, fact provider service, and/or other rules, workflow and/or event providing service (e.g., Drools engine, etc.).

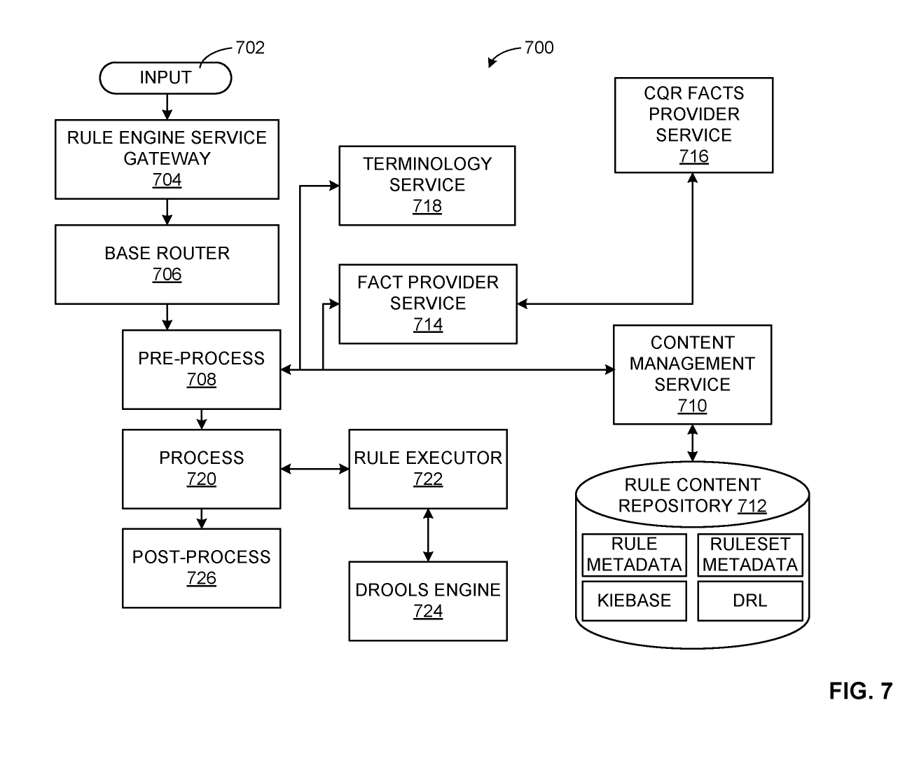

FIG. 7 illustrates a flow diagram of an example rules engine execution flow 700 among rules engine 640 components to build and execute a rule for a given input. For example, one or more rules can be built and executed to facilitate patient and practice management for patient search, patient registration, virtual office visits, schedule management, patient intake, eligibility management, patient liability estimation, fee management, system and operability, etc.

At block 702, an input is provided to the rules engine 640 to generate an output. The example input 702 can include one or more of a rule set identifier, a tenant identifier, a specialty identifier, a user identifier, an “as of” date, fact(s), etc. The generated output can include a list of actions, for example. A rule engine service gateway 704 provides the input 702 to a base router 706.

At block 708, the data is pre-processed. For example, the input 702 can be processed with a fact provider service 714 to verify and/or supplement fact(s) provided in the input and/or provide fact(s) (e.g., from a Clinical Quality Reporting (CQR) fact provider service 816, etc.) in response to identifier(s) provided in the input. A terminology service 718 provides and/or adjusts proper terminology for a rule in accordance with the fact(s) and/or identifier(s) provided in the input 702. Pre-processing 7u08 can also include accessing a rule content repository 712 via a content management service 710. The example rule content repository 712 includes rule metadata, ruleset metadata, a Drools rule language (DRL) core, Kiebase and/or other application knowledge definition repository, etc.

Retrieved rule, fact, and/or terminology information is provided with the input 702 for processing at block 722. During processing 722, a rule executor 722 calls a rules execution engine such as a Drools engine 724 to execute the rule based on provided fact(s), identifier(s), metadata, etc. The result is provided for post-processing at block 726 (e.g., to clean up, frame, present, and/or otherwise prepare result(s) for output, etc.).

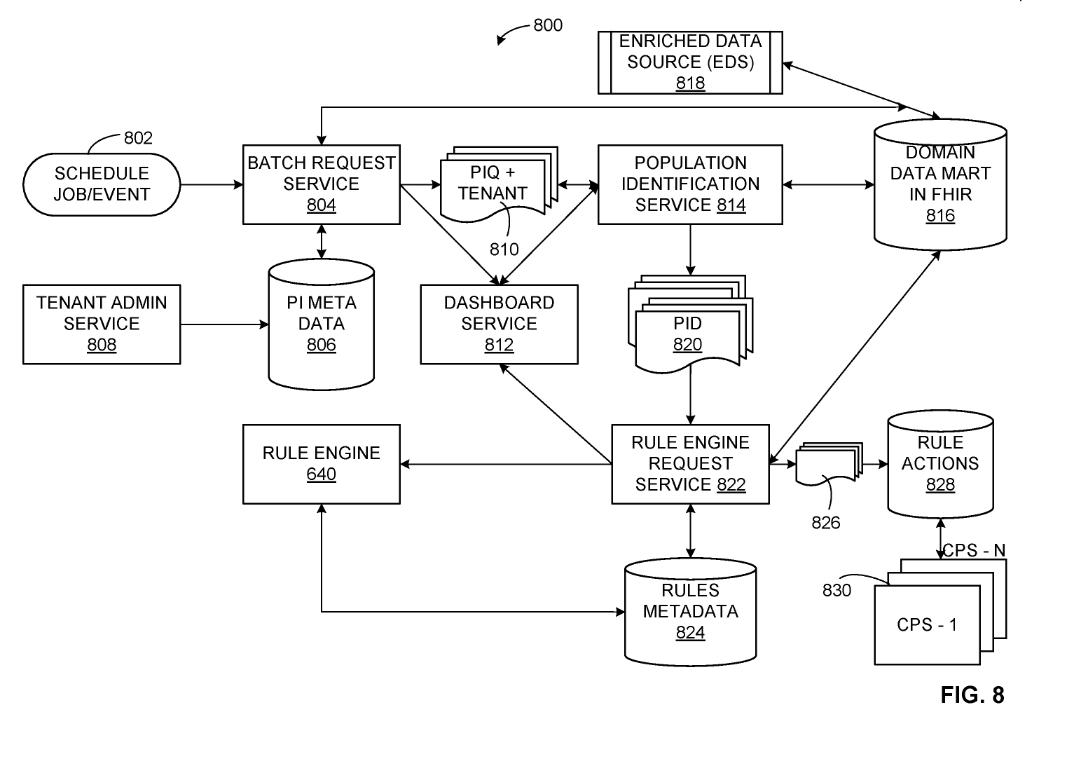

FIG. 8 illustrates a flow diagram of an example method 800 for a batch process flow to provide recommendation and/or other data to a clinician based on patient history and facts. For example, the batch process for clinical decision support helps in analyzing thousands of patients during a non-peak time to generate recommendations in advance of a physician-patient encounter. Services included in the batch flow help provide a workflow to identify patients, executed rules, and persist recommendations. For patient-focused applications, such as a patient navigation interface, the batch process flow can be used to facilitate virtual visits, real-time scheduling, payment, and patient enablement through mobile access via the interface.

At block 802, a job or event is scheduled based on a trigger generated from an update of domain data. The scheduled job/event is provided to a batch request service 804 with associated metadata for a given tenant (e.g., user, job, event, etc.). The batch request service 804 communicates with patient identifier metadata 806. A tenant administrator service 908 works with the patient identifier metadata 806 to generate a configuration for each tenant. The configuration includes metadata 806 such as tenant, patient identification and qualification (PIQ), ruleset, etc.

In certain examples, the batch flow is provided via a cloud-based application to service hundreds of tenants such as hospitals, ambulatory care centers, etc. For each tenant, a PIQ message is placed in a Patient Identifier Request (PIR) queue 810 with a tenant identifier, which can be used by downstream components for further processing. Information can also be provided to a dashboard service 812 for analysis and display.

The PIR is provided from the queue 810 to a population identification service 814. The population identification service 814 can be implemented, for example, as a REST service that can be invoked by another service, component, etc. The population identification service 814 watches the PIR queue 810 and processes received messages. For each tenant, the service 814 identifies patients (e.g., all patients or a subset created using a filter criterion to select patients based on appointment date, age, other criterion, etc.), and an individual message is created and placed in the rule execution queue 820.

Thus, the population identification service 814 constructs a query and pulls a patient identifier (PID) from a domain data mart 816 (e.g., a Fast Health Interoperability Resources (FHIR) domain data mart, etc.) which includes patient data from one or more enriched data sources (EDS) 818, for example. In certain examples, the data mart 816 runs with a patient FHIR service to provide patient information in the FHIR model. The population identification service 814 can use the FHRI model and associated service to fetch details for a patient. After retrieval, the population identification service 814 places the PID into a rule execution request queue 820.

For example, once the request is successfully processed, an entry is made on a service bus based PIR queue 820. The rule engine request service 822 pulls a message from the rule execution queue 820 and extracts metadata from a rules metadata 824. The request service 822 creates an input from the PID and metadata and facts retrieved from the data store 816. The request service 822 provides the input to the rules engine 640. The rules to be run as part of the batch process can be configured using property settings, for example. Rules can be tenant-specific, tenant-agnostic, etc.

For each identified patient, patient facts are pulled from an external repository 816 and then fed to the rules engine 640. The rules engine processes the data and provides one or more recommendations, which is/are then persisted via a rules result service 826. The rules result/action service 826 persists recommended actions based on execution of the rule with respect to the patient. The service 826 persists or stores recommended actions(s) as records 830 in a rules results repository 828. In certain examples, the recommendations are split into single actions so that independent processing can be performed against each action. If there are no recommendations are provided in the rules response, a record 830 with an empty action can be persisted, for example.

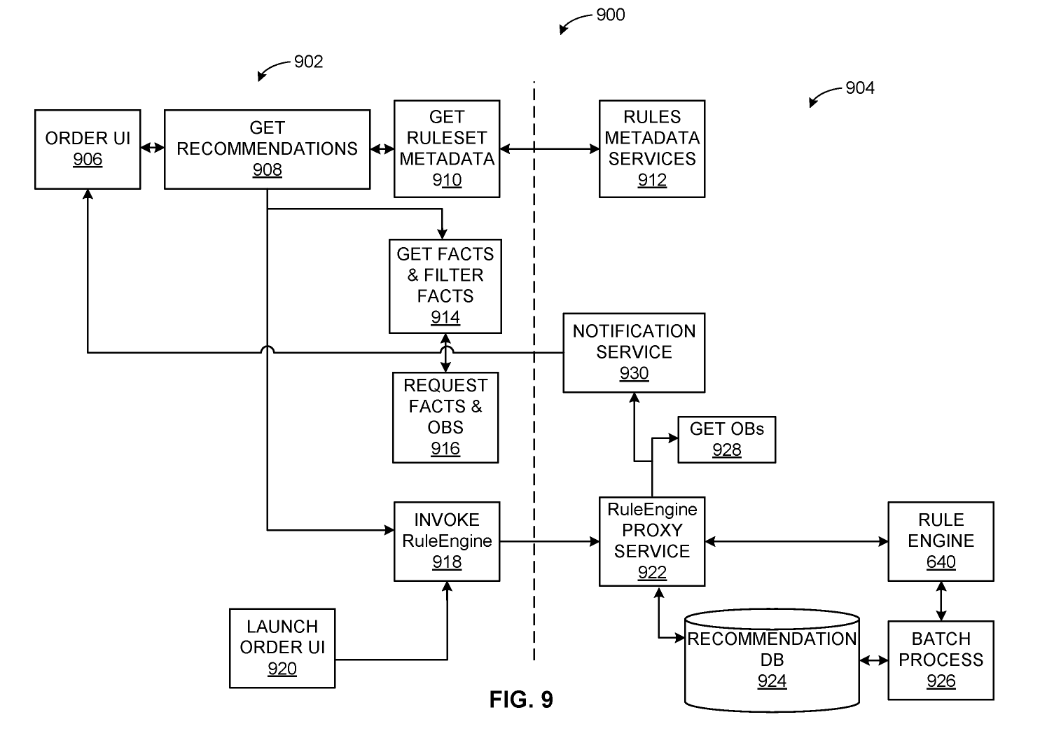

In certain examples, the rules engine 640 works with and/or is integrated with a clinical practice management system and/or other healthcare information system. As shown in the example system 900 of FIG. 9, the rules engine 640 can be located in the cloud 902 and interact with a local, on-premise information system 904.

At block 906, an order user interface (UI) can be used to add a problem (e.g., associated with a patient) via the on-premise information system 902 (e.g., a practice management system, electronic medical record, etc.). At block 908, recommendation(s) for rule formation are retrieved from the information system 902. At block 910, ruleset metadata is retrieved. After retrieving the ruleset metadata, the ruleset is provided to a rules metadata services 912 in the cloud 904.

At block 914, facts are requested and filtered. For example, patient information and/or other facts relating to rules are requested and filtered according to patient, ruleset relevance, etc. At block 916, facts and observations are requested and retrieved (e.g., from the on-premise information system 902) for filtering at block 914. At block 918, the rule engine 640 is invoked using the facts and rules. At block 920, the order UI can be launched asynchronously to invoke the rule engine 640 (block 918) and batch recommendation via the on-premise information system 902, for example.

At block 922, a proxy service for the rule engine 640 facilitates invocation of the rule engine 640 via the cloud 904. The proxy service accesses a recommendation database 924 and batch process 926 in conjunction with the rule engine 640. The rule engine 640 provides a recommendation in the recommendation database 924 for one or more rulesets and facts via the batch process 926. The proxy service 922 can retrieve observation information 928 and leverage a notification service 930 to provide an output via the user interface 906.

Thus, patient facts can be made available in the on-premise information system 902 (e.g., practice management system client cache, etc.) and can be filtered. Requests and recommendations can be batched and executed with the rule engine 640 via the cloud 904 to generate a notification for user(s) generating an order for a patient (e.g., including users who have navigated away from the order screen, etc.).

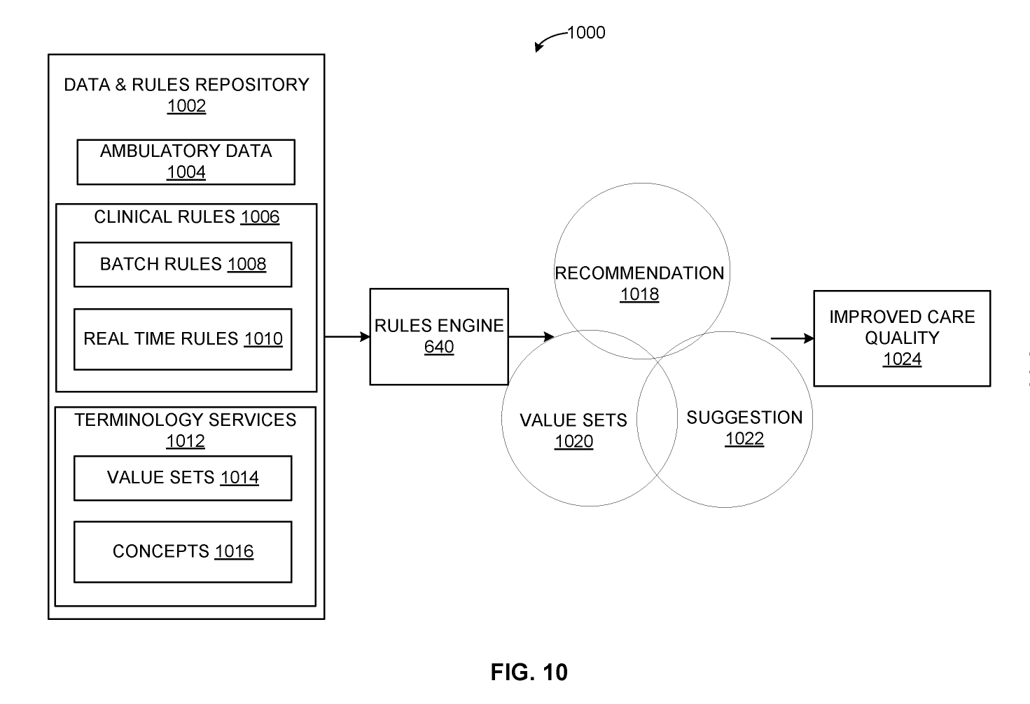

FIG. 10 illustrates an example clinical decision support system 1000 including a data and rules repository 1002. The example repository 1002 includes ambulatory data 1004, clinical rules 1006, and terminology services 1012. The example ambulatory data 1004 includes information such as patient identifier, procedure identifier, order, observation, medication order, medication, immunization, care goal, family history, encounter, problem, allergy intolerance, etc. The rules engine 640 leverages the ambulatory data 1004 using rules 1006 and terminology services 1012. The example clinical rules 1006 include batch rules 1008 and real time rules 1010 (see, e.g., the description of FIGS. 4-10). The example terminology services 1012 includes value set(s) 1014 and concept(s) 1016 to support the interpretation of data 1004 according to the rules 1006 by the rules engine 640, for example. The rules engine 640 generates a recommendation 1018, a value set 1020, and/or a suggestion 1022 to facilitate improved care quality 1024.

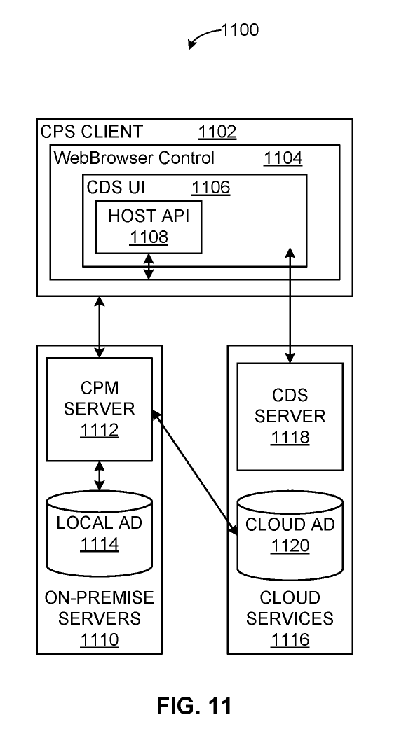

In certain examples, the rules engine 640 and associated repository 1002 can be embedded in one or more other clinical applications 1100. For example, as shown in FIG. 11, a CPS client 1102 includes a web browser control 1104, a clinical decision support (CDS) user interface 1106, and a host API 1108. The CPS client 1102 communicates with one or more on-premise servers 1110 and one or more cloud-based and/or otherwise remote servers 1116. Each on-premise server 1110 includes a clinical practice management (CPM) server 1112 and a local active directory (AD) 1114. Each cloud-based server 1116 includes a clinical decision support (CDS) server 1118 and a cloud AD 1120. The cloud AD 1120 can provide information to the CPM server 1112, for example.

Thus, a user logs in via the CPS client 1102, and the CPM server 1112 can authenticate the user via the local AD 1114 as well as the cloud AD 1120. The user invokes orders, and the CPM server 1112 loads the clinical decision support 1118 address (e.g., uniform resource locator (URL), etc.) in the Web Browser control 1104 for interaction via the UI 1106. The rules engine 640 and repository can be leveraged by the user from the CDS server 1118 via the UI 1106, for example.

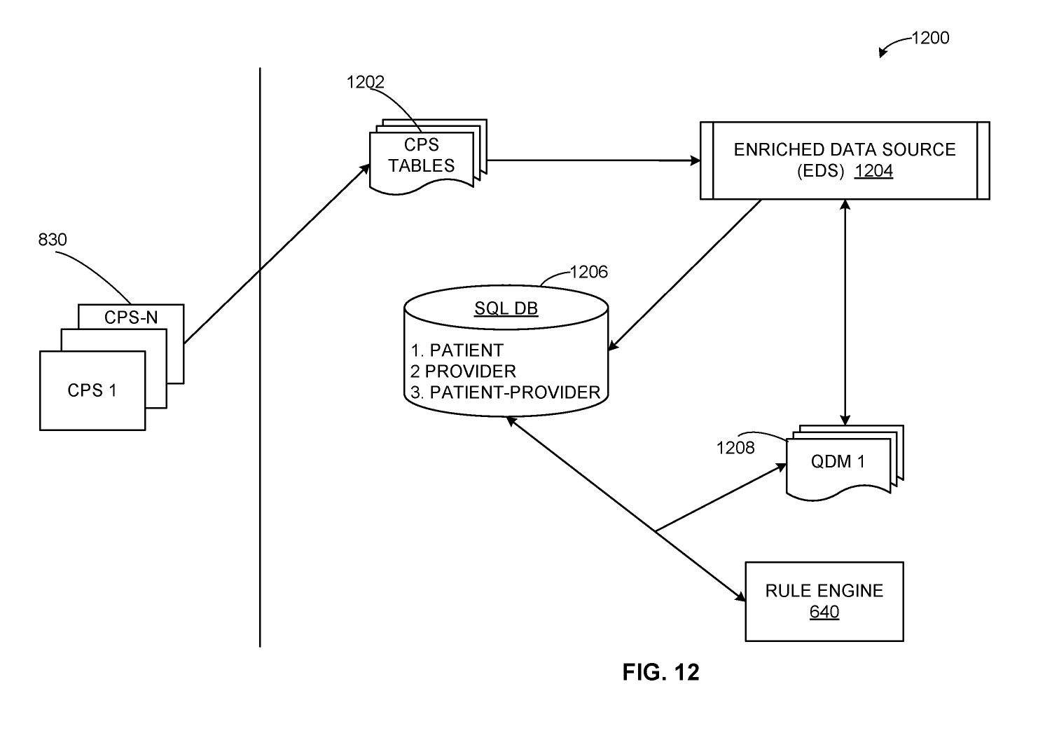

FIG. 12 provides a data flow 1200 for clinical quality reporting (CQR) using the rule engine 640. As shown in the example of FIG. 12, data is moved from one or more on-premise CPS 830 to one or more CPS tables 1202 located in the cloud via an application development framework (ADF), for example. Enriched data is created with standard codes and provided from the CPS table(s) 1202 to an enriched data source (EDS) 1204. Enriched data in the EDS 1204 can be formed using term mapping and can be partitioned by patient identifier, tenant, etc. The EDS 1204 can be implemented in a data warehouse system such as a Hive data warehouse, etc., with data organized according to one or more quality data models (QDM), for example.

Enriched data can be executed from the EDS 1204 to a database 1206, such as a structured query language (SQL) database 1206 according to patient, provider, and patient-provider information (e.g., using one or more SQL tables to identify patients, etc.). Additionally, a patient longitudinal health record (LHR) can be formed from QDMs 1208 of enriched patient data from the EDS 1204. In certain examples, the database 1206 can be queried to identify patient(s), and corresponding patient data can be pulled from the QDM 1208. The rule engine 640 is invoked for each patient QDM 1208 to process patient data according to rule(s) to generate a clinical quality report, for example.

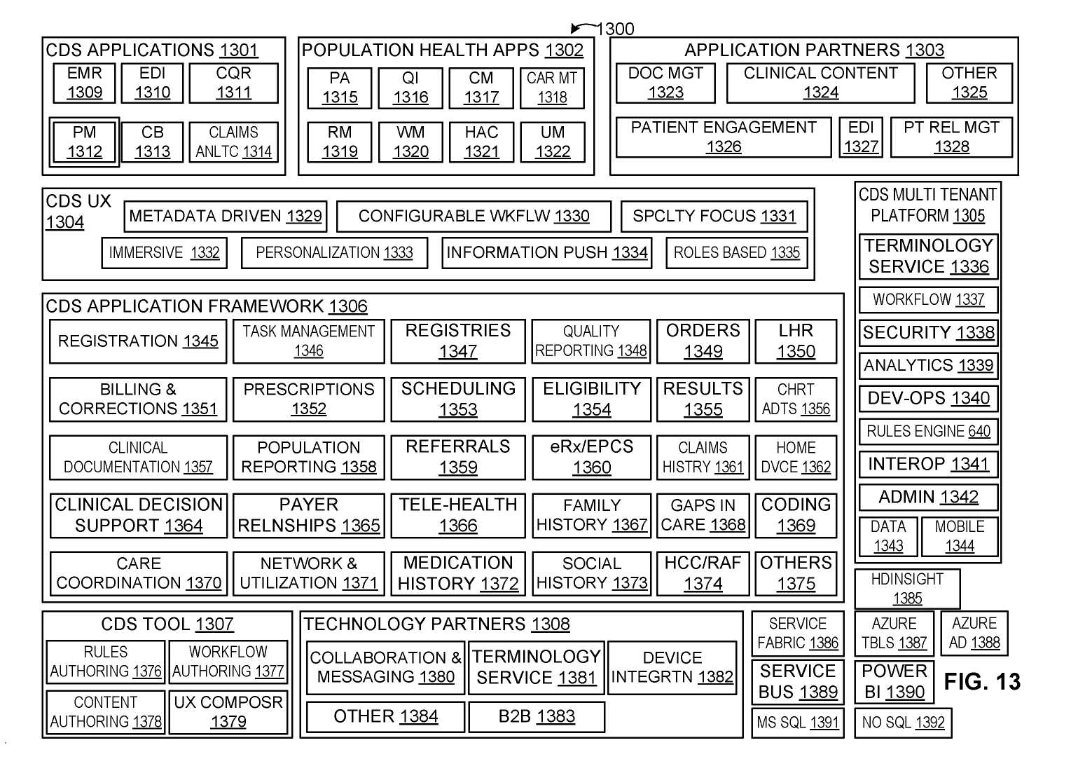

FIG. 13 illustrates an architectural block diagram of an example clinical decision support (CDS) system 1300 including the rules engine 640. The example CDS system 1300 includes CDS applications 1301, population health applications 1302, partner applications 1303, CDS user experience (UX) 1304, a CDS multi-tenant platform 1305, a CDS application framework 1306, CDS tools 1307, technology partners 1308, etc.

More particularly, as shown in the example of FIG. 13, CDS applications 1301 can include an EMR 1309, electronic data interchange (EDI) 1310, CQR 1311, practice management (PM) 1312, clinical business (CB) 1313, claims analytics (e.g., GE Denials-IQ™, etc.) etc. Population health applications 1302 can include patient-provider assignment (PA) 1315, quality improvement (QI) 1316, care management (CM) 1317, care management 1318, risk management (RM) 1319, patient wellness management (WM) 1320, hospital acquired condition (HAC) 1321, utilization management (UM) 1322. Other partner applications 1303 can include document management 1323, clinical content 1324, other content 1325, patient engagement 1326, EDI 1327, patient relationship management 1328, etc.

Additionally, as shown in the example of FIG. 13, CDS UX 1304 components can include a metadata driven UX component 1329, a configurable workflow 1330, a specialty focus UX component 1331, an immersive UX component 1332, a personalization component 1333, an information push component 1334, a role-based UX component 1335, etc. The example CDS multi-tenant platform 1305 can include a terminology service 1336, a workflow engine 1337, a security component 1338, analytics 1339, a development and operations (dev-ops) component 1340, the rules engine 640, an interoperability component 1341, an administrator 1342, a data component 1343, a mobile component 1344, etc.

In the example of FIG. 13, the CDS application framework 1306 can include registration 1345, task management 1346, registries 1347, quality reporting (QR) 1348, orders 1349, longitudinal health record (LHR) 1350, billing and corrections 1351, prescriptions 1352, scheduling 1353, eligibility 1354, results 1355, chart audits 1356, clinical documentation 1357, population reporting 1358, referrals 1359, electronic prescriptions (eRx)/electronic prescriptions for controlled substances (EPCS) 1360, claims history 1361, home device 1362, clinical decision support (CDS) 1364, payer relationships 1365, tele-health 1366, family history 1367, gaps in care 1368, coding 1369, care coordination 1370, network and utilization 1371, medication history 1372, social history 1373, hierarchical condition category (HCC)/risk adjustment factor (RAF) 1374, other 1375.

In the example of FIG. 13, the CDS tools 1307 can include a rules authoring tool 1376, a workflow authoring tool 1377, a content authoring tool 1378, a UX composer tool 1379, etc. Technology partners 1308 can include collaboration and messaging 1380, a terminology service 1381, device integration 1382, business-to-business (B2B) transaction component 1383, and/or other component 1384, for example. Other components in the example system 1300 include a service fabric 1386, cloud (e.g., Microsoft Azure™, etc.) tables 1387, cloud active directory 1388, a service bus 1589, business analytics (e.g., Microsoft Power BI, etc.) 1390, data server (e.g., Microsoft SQL server, etc.) 1391, non-relational database 1392 (e.g., NoSQL, etc.), etc. Thus, the rules engine 640 can be used with a variety of applications 1301, 1302, 1303, 1306, 1308 as well as authoring tools 1307 and user interface components 1304 to provide rules-based application services to a user.

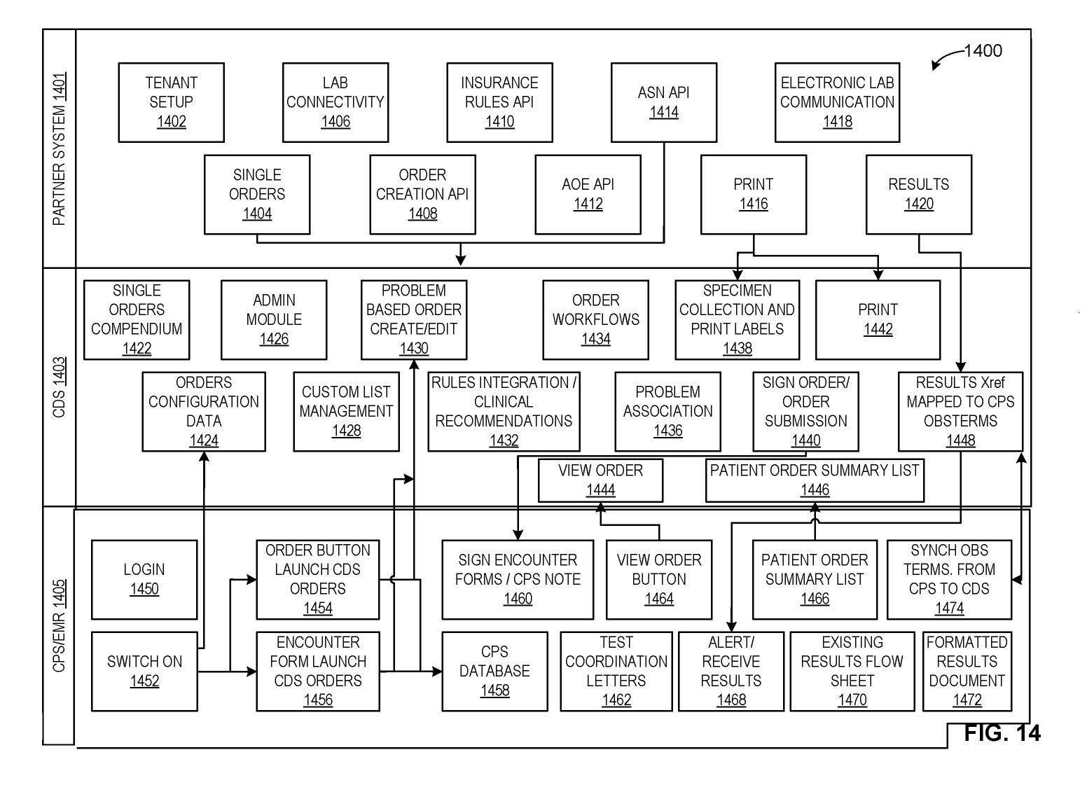

FIG. 14 illustrates another example block diagram of a system 1400 integrating a partner system 1401 with a CDS 1403 and CPS/EMR 1405. As shown in the example of FIG. 14, the partner system 1401 can provide one or more API, communication, etc., to interface with the CDS 1403 which interfaces with the CPS/EMR 1405 to leverage stored clinical information and rules to deliver application functionality to a user.

As shown in the example of FIG. 14, partner system 1401 components such as tenant setup 1402, single orders 1404, lab connectivity 1406, order creation API 1408, insurance rules API 1410, Ask at Order Entry (AOE) API 1412, abstract syntax notation (ASN) API 1414, print component 1416, electronic lab communication 1418, results 1420, etc. Order(s) 1404 can be provided to the CDS 1403 for rules-based processing.

For example, the CDS 1403 provides a single orders compendium 1422 to receive the single order 1404. Order configuration data 1424 provides order configuration information to the administrative module 1426 and custom list management 1428. The CDS 1403 provide problem-based order creation and/or editing 1430 as well as rules integration/clinical recommendation 1432. The rules integration/clinical recommendation component 1432 can include the rules engine 640 and its associated repository, terminology services, etc., for example. The CDS 1403 also provide order workflows 1434 and problem association 1436. The CDS 1403 includes specimen collection and print labels 1438 and print component 1442 to interact with the print component 1416 of the partner system 1400. An order signature/order submission component 1440 interacts with the CPS/EMR 1405 to submit an order. The order can be viewed 1444 and a patient order summary list 1446 can be provided. The results 1420 from the partner system 1401 are cross-referenced and mapping to observation terms at the CPS/EMR 1405 via the mapper 1448 of the CDS 1403.

As shown in the example of FIG. 14, the CPS/EMR 1405 provides a login 1450 and switch on or activator 1452 to access order configuration data 1424 from the CDS 1403 and/or launch CDS orders via an order button 1454 and/or encounter form 1456. Order(s) are provided to a CPS database 1458. The sign order/order submission 1440 at the CDS 1403 communicates with an encounter form/note sign component 1460 at the CPS/EMR 1405. Test coordination letter(s) 1462 can be generated by the CPS/EMR 1405. The CPS/EMR 1405 can provide a view order button 1464 for a user to view order(s) 1444 stored at the CDS 1403. A patient order summary list 1466 can be provided by the CPS/EMR 1405 to the patient order summary list 1446 of the CDS 1403. The CPS/EMR 1405 can communicate with the mapper 1448 of the CDS 1403 to generate an alert/receive results 1468, for example. Results can be used to generate a flow sheet 1470. The flowsheet 1470 can be used to generate a formatted results document 1472. Observation terms can be synchronized 1474 between the mapper 1448 at the CDS 1403 and the CPS/EMR 1405.

Thus, certain examples provide systems and methods in which workflow rules drive automation for electronic medical records and other systems through tasking, alerts, suggestions, etc. Certain examples facilitate completion of various portions of medical practice activities and/or patient review/access (e.g., management, registration, rooming, encounter, visit summary, billing, etc.) through facilitation of the workflow to eliminate manual processes. Certain examples provide a role-based rules system to determine access, tasks, alerts and suggestions to be presented to complete medical practice activities. In certain examples, the system also allows for both role-based rules and individual custom rules to be created. Certain examples drive web page and/or other interface content navigation to complete tasks.

Using the rules engine 640, for example, provider input and/or one or more triggers can activate the rules engine 640 to apply productive analytics. An example of such analytics is as follows: Suppose a patient is over the age of 13; the rules engine 640 can advise a provider to inquire whether the patient smokes. Such alerting and proactive suggestion for provider-patient encounter interaction, diagnosis, and/or treatment can be extrapolated into more complex conditions such as cancers and/or other inherited conditions. For example, if the patient has a documented family history, the rules engine 640 can advise the provider to inquire further and advise productive procedures such as a mammogram for breast cancer. Analytics, such as meaningful use (MU), clinic-specific, custom, etc., can be built, executed, and maintained in conjunction with the rules engine 640 to provide clinical decision support.

Patients and providers have a limited amount of time and cognitive energy. By providing a solution that decreases the amount of cognitive load and time required for initial and mundane diagnostics, providers can instead exert their cognitive energy on more complex conditions. Additionally, patient outcomes can be improved by intervening earlier in the patient care cycle. Certain examples enable a provider to provide benchmark goals to the patient to prevent worsening of a condition and improve overall health while decreasing medical cost over the patient's life time. Certain examples facilitate patient proactivity and interaction with the healthcare process.

Certain examples, increase medical evidence if a patient does not comply with preventive measures. Insurance claims can become more accurate due to greater and more complete sets of monitored information.

In certain examples, machine learning technologies (e.g., convolutional neural network, deep learning network, feature-based machine learning, etc.) can be applied to unlabeled patient data in one or more specified subject areas such as smoking, drinking, etc. The system can apply the machine learning to determine points of intervention between the provider and his or her patient.

In operation, in certain examples, the rules engine 640 executes against patient facts and type of appointment scheduled. The clinical decision support system packages results as tasks, activities and/or order recommendations to end users for next steps in patient care (e.g., in a patient care plan or pathway, etc.).

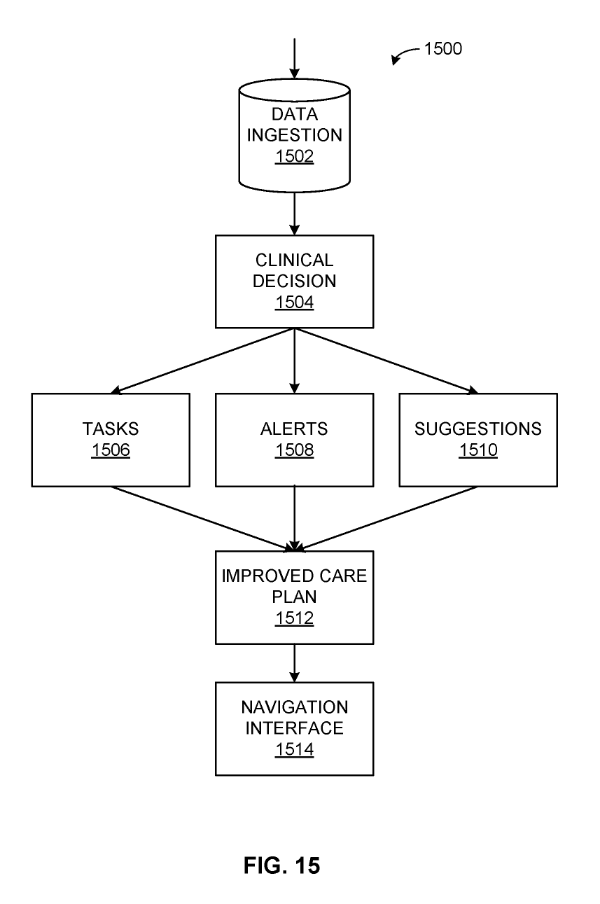

Thus, as shown in the example of FIG. 15, a clinical decision support system 1500 can leverage the rules engine 640 as part of the clinical decision support to generate an improved patient outcome. As shown in the example of FIG. 15, data ingestion 1502 receives and processes (e.g., formats, normalizes, validates, etc.) incoming data such as patient data, purpose/reason for examination, lab results, etc. Clinical decision support 1504 (e.g., including the rules engine 640, EMR, scheduling/billing system, etc.) processes the ingested data based on one or more rules, repository information, etc., to generate one or more tasks 1506, alerts 1508, and/or other suggestions 1510 for the patient. The tasks(s) 1506, alert(s) 1508, and/or suggestion(s) 1510 are used to generate an improved care plan 1512 for the patient.

In certain examples, clinical decision support 1504 can involve adjustment of a rule and/or creation of a new rule via the rules engine 640 and repository based on the ingested information for the patient. Monitoring of incoming clinical data (e.g., from patient appointments, lab results, and/or other input data, etc.) can be used with respect to the rule(s) to determine whether the patient care plan 1512 is being followed to complete a specified task 1506, for example. If the task 1506 is not being accomplished according to the care plan 1512, an alert 1508 can be generated and/or a suggestion 1510 can be provided to the patient, provider and/or to adjust the task 1506 and/or overall care plan 1512 based on the alert 1508 and/or suggestion 1510, for example. A navigational interface 1514 can output the alert 1508 as well as task 1506 information, care plan 1512 information, and/or other information and/or functionality to a patient and/or provider user, for example.

Thus, certain examples enable ingestion of information and evaluation of rule(s) to generate, monitor, prompt, and adjust a patient care plan. Certain examples facilitate monitoring of patient data and comparison to evaluate compliance with the care plan and satisfaction of a goal for the patient. In certain examples, the care plan and/or goal are modified if the goal is not being attained and/or the care plan is not being followed.

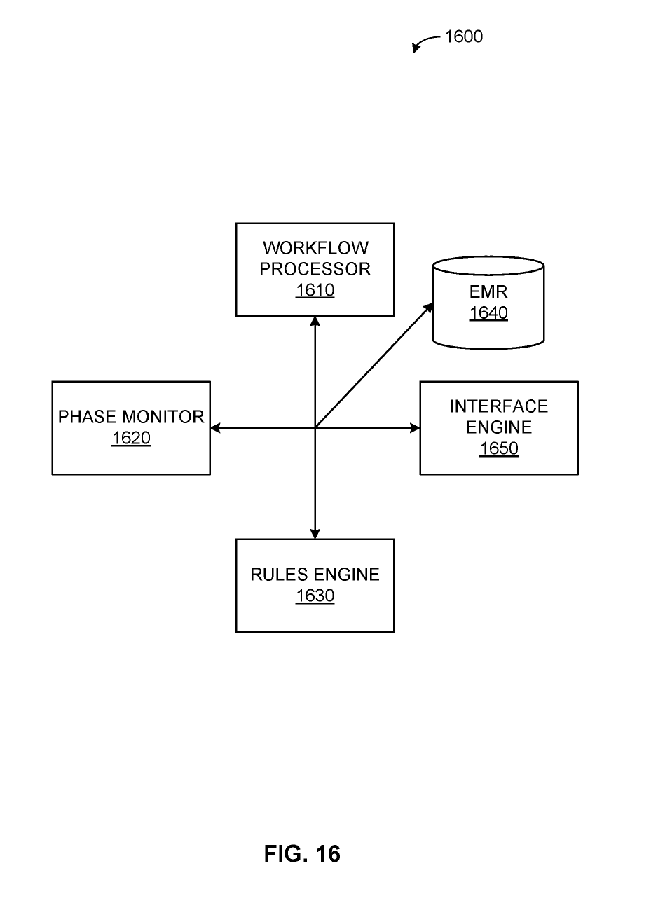

FIG. 16 illustrates an example system 1600 implementing the rules engine 640 and clinical decision support 1403 in the form of a workflow processor 1610, a phase monitor 1620, a rules engine 1630, an EMR 1640, and an interface engine 1650. For example, the CDS 1403 can be used to form the workflow processor 1610 and/or the phase monitor 1620, the rules engine 640 can be used to form the rules engine 1630, and the EMR/CPS 1405 can be used to form the EMR 1640.

In an example, the rules engine 1630 and the EMR 1640, in conjunction with the workflow processor 1610, generate a workflow to treat a particular patient according to information regarding that patient (e.g., patient exam records, diagnosis of condition, rules and/or other protocol information regarding the condition, etc.). The example workflow processor 1610, rules engine 1630, and EMR 1640 identify roles relevant to the workflow in the healthcare environment. The workflow processor 1610 divides the workflow into phases. Each phase includes at least one activity specified by rule(s) from the rules engine 1630. Each activity involves at least one role and at least one associated task, alert, and/or suggestion for the at least one role specified by the rule(s). The workflow processor 1610 associates each phase with its rule(s) and role(s). The rules from the rules engine 1630 connect the activities of the phases of the workflow to the EMR 1640 automatically provide and/or receive data for the patient in each phase of the workflow.

The example workflow processor 1610 facilitates execution of the workflow with respect to the patient. For example, the workflow processor 1610 facilitates interaction between healthcare systems, the patient, and/or a healthcare provider to executes the workflow associated with care of the patient (e.g., a patient care plan, care path, treatment protocol, etc.). The example phase monitor 1620 monitors execution of the workflow to determine which phase of the workflow is executing. The rules engine 1630 can then work with the phase monitor 1620 and the workflow processor 1610 to determine successful and/or unsuccessful execution of activities or tasks in the workflow phase. An identified deviation from the workflow phase can result in an alert (e.g., to the patients, the provider, and/or healthcare system(s), etc.), a suggestion, a task adjustment, etc. Execution of the workflow and its phases can be monitored and adjusted dynamically in real time (or substantially real time given information transmission and/or processing delay) using the phase monitor 1620 and the workflow processor 1610 in conjunction with rules from the rules engine 1630 and data from the EMR 1640.

The workflow processor 1610, phase monitor 1620, rules engine 1630, and EMR 1640 can drive content and functionality for the interface engine 1650. For example, the interface engine 1650 works in conjunction with the workflow processor 1610 to facilitate interaction between healthcare systems, the patient, and/or a healthcare provider to executes the workflow associated with care of the patient (e.g., a patient care plan, care path, treatment protocol, etc.). The interface engine 1650 generates a representation of current workflow phase (as well as prior and/or next workflow phase, etc.) based on information from the phase monitor 1620. The interface engine 1650 can also generate an indication of successful and/or unsuccessful execution of activities or tasks in the workflow phase. The interface engine 1650 can generate a visual alert via a GUI triggered in response to an identified deviation from the workflow phase, for example. The interface engine 1650 can surface information such as activity and associated task(s) via the GUI based on user role, workflow phase, etc.

Thus, the workflow processor 1610, phase monitor 1620, rules engine 1630, EMR 1640, and interface engine 1650 identify the patient, identify the workflow for patient care, identify personnel and resources (e.g., assets) to care for the patient through the workflow, divide workflow into phases based on activity and role, monitor execution of phases, and provide information to/from assets to drive the workflow for care of the patient.

Certain examples facilitate creation of role-based rules and/or custom rules using the rules engine 1630, EMR 1640 and/or feedback gathered from the workflow processor 1610 and/or phase monitor 1620. Certain examples provide a cloud-based platform leveraged through interaction with system APIs to support roles and functions for workflow(s). In certain examples, in addition to patient diagnosis and/or treatment activities, medical practice activities. Medical practice activities can include management, registration, rooming, encounter, visit summary, billing, etc. Certain examples provide an integrated solution including database(s), information technology infrastructure, user interface, task management, front end operation, etc. In certain examples, a journey map can be formed for a patient and/or provider including roles (e.g., provider role(s), other persona(s), etc.), resources (e.g., assets and/or other components, services, etc.), activities/tasks, and associated rules for each phase of the healthcare workflow. The journey map can be displayed via the interface engine 1650. In certain examples, rules can be changed dynamically on the cloud-based server side, but roles and phases remain unchanged on the user end in the healthcare environment.

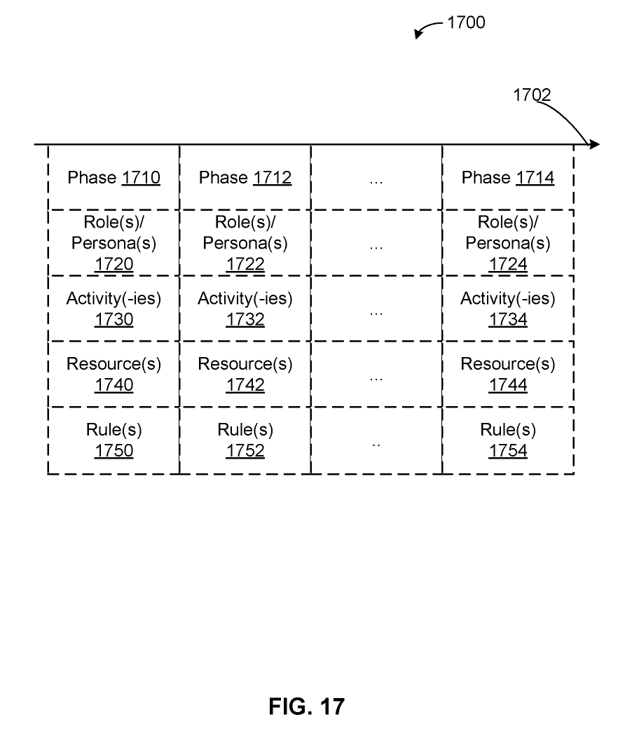

FIG. 17 illustrates a visualization of a journey map 1700 for a healthcare workflow 1702 for a patient. The patient workflow or journey 1702 is divided into phases 1710-1714. Within each phase 1710-1714, one or more roles/personas 1720-1724 are allocated to the respective phase 1710-1714. Thus, the map 1700 of the workflow 1702 provides an indication of who is to be involved in each phase 1710-1714 based on the included role(s) 1720-1724.

Further, each phase 1710-1714 includes an indication of activity(-ies) 1730-1734 involved in the respective phase 1710-1714. Thus, for each phase 1710-1714, the map 1700 informs which role(s) 1720-1724 are to perform which task(s) 1730-1734 in that phase 1710-1714. Additionally, the map 1700 identifies resource(s) 1740-1744 used in the activity(-ies) 1730-1734 for the role(s) 1720-1724 in each phase 1710-1714. The journey map 1700 also includes an indication of rule(s) 1750-1754 governing the activity(-ies) 1730-1734 and/or associated role(s) 1720-1724 and/or resource(s) 1740-1744 involved in each phase 1710-1714.

Thus, the example journey map 1700 for the workflow 1702 can be used to drive the workflow 1702 such that the workflow processor 1610, phase monitor 1620, rules engine 1630, EMR 1640 and associated role(s) 1720-1724 understand where, when, and how the phases 1710-1714 of the workflow 1702 should be executed. For example, phases 1710-1714

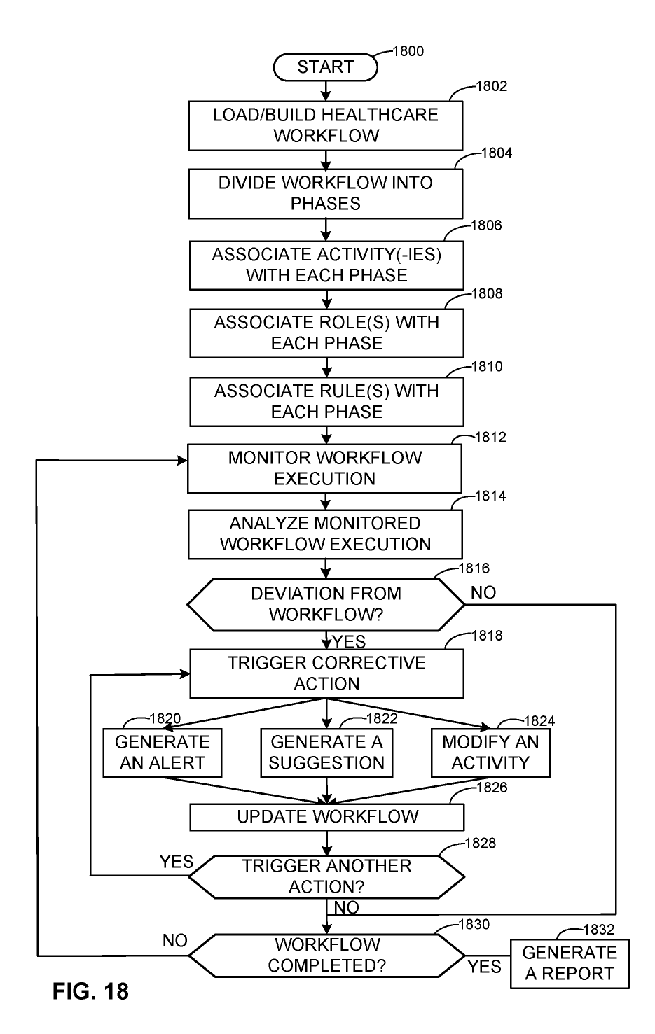

FIG. 18 illustrates a flow diagram of an example method 1800 of patient care plan composition, monitoring, and adjustment using the example workflow processor 1610, phase monitor 1620, rules engine 1630, and EMR 1640. At block 1802, a healthcare workflow is loaded and/or built for processing in preparation of execution. For example, the rules engine 1630 and the EMR 1640, in conjunction with the workflow processor 1610, load and/or build a workflow to treat a particular patient according to information regarding that patient (e.g., patient exam records, diagnosis of condition, rules and/or other protocol information regarding the condition, etc.).

Then, the workflow is processed. At block 1804, the workflow is divided into phases. The workflow processor 1610 divides the workflow into phases, segments, or portions representing discrete parts of the healthcare workflow to be executed with respect to the patient. At block 1806, one or more activities/tasks are associated with each phase of the workflow. For example, each phase includes at least one activity (e.g., registration, pre-exam, exam, post-exam, follow-up, pre-op, operation, post-op, etc.) specified by the workflow. The workflow processor 1610 identifies and associates activity(-ies) with each phase of the workflow.

At block 1808, one or more roles (e.g., patient, receptionist, nurse, primary physician, radiologist, surgeon, imaging technician, billing clerk, other persona, etc.) are associated with each phase of the workflow. The workflow processor 1610 associates each phase with its role(s). At block 1810, one or more rules are associated with each phase of the workflow. Each activity involves at least one role and at least one associated task, alert, and/or suggestion for the at least one role specified by the rule(s). The workflow processor 1610 associates each phase with its rule(s) and role(s). The rules from the rules engine 1630 connect the activities of the phases of the workflow to the EMR 1640 automatically provide and/or receive data for the patient in each phase of the workflow. Thus, the example workflow processor 1610, rules engine 1630, and EMR 1640 identify activities, roles, and rules relevant to the workflow in the healthcare environment and instantiates them with respect to each phase.

At block 1812, execution of the workflow by the workflow processor 1610, rules engine 1630, and EMR 1640 is monitored by the phase monitor 1620. The example the workflow processor 1610 facilitates execution of the workflow with respect to the patient. For example, the workflow processor 1610 facilitates interaction between healthcare systems, the patient, and/or a healthcare provider to executes the workflow associated with care of the patient (e.g., a patient care plan, care path, treatment protocol, etc.). The example phase monitor 1620 monitors execution of the workflow to determine which phase of the workflow is executing and how.

At block 1814, monitored execution of the workflow is analyzed by the phase monitor 1620 to determine a deviation from the workflow. For example, the rules engine 1630 can work with the phase monitor 1620 and the workflow processor 1610 to determine successful and/or unsuccessful execution of activities or tasks in the workflow phase. At block 1816, a deviation from the workflow is determined. If a deviation is identified, then, at block 1818, one or more corrective actions are triggered.

For example, an identified deviation from the workflow phase can result in an alert (e.g., to the patients, the provider, and/or healthcare system(s), etc.), a suggestion, a task adjustment, etc. At block 1820, an alert is generated in response to the deviation from the workflow. For example, an audible and/or visible alert is generated for the provider, the patient, etc. In certain examples, an electronic alert can be logged, routed to another program, etc. At block 1822, a suggestion of action is generated in response to the deviation from the workflow. For example, a tip, suggestion, reminder, etc., can be generated for the patient and/or provider to help comply with the workflow (e.g., exercise, reposition, register, fast, add more contrast, etc.). At block 1824, an activity in the phase of the workflow is modified. For example, an amount of exercise, a period of fasting before labs are taken, a dosage of medication, a time for surgery, etc., can be modified to adjust for the deviation from the expected workflow. In certain examples, a change in care plan and the associated workflow can occur through a change in medication, an ordering of a new/different exam, a request for additional labs, an instruction for different eating habits and/or exercise, etc.

At block 1826, the workflow is updated based on the triggered action. At block 1828, if more than one corrective action was indicated, then control returns to block 1818 to trigger an additional corrective action. If no further corrective action is indicated, then, at block 1830, the workflow is examined to determine if the workflow has been completed. If the workflow has not been completed, then control returns to block 1812 to monitor execution of the workflow. If the workflow has been completed, then, at block 1832, a report is generated to capture the patient and/or provider's journey through the workflow and its execution. Thus, execution of the workflow and its phases can be monitored and adjusted dynamically in real time (or substantially real time given information transmission and/or processing delay) using the phase monitor 1620 and the workflow processor 1610 in conjunction with rules from the rules engine 1630 and data from the EMR 1640.

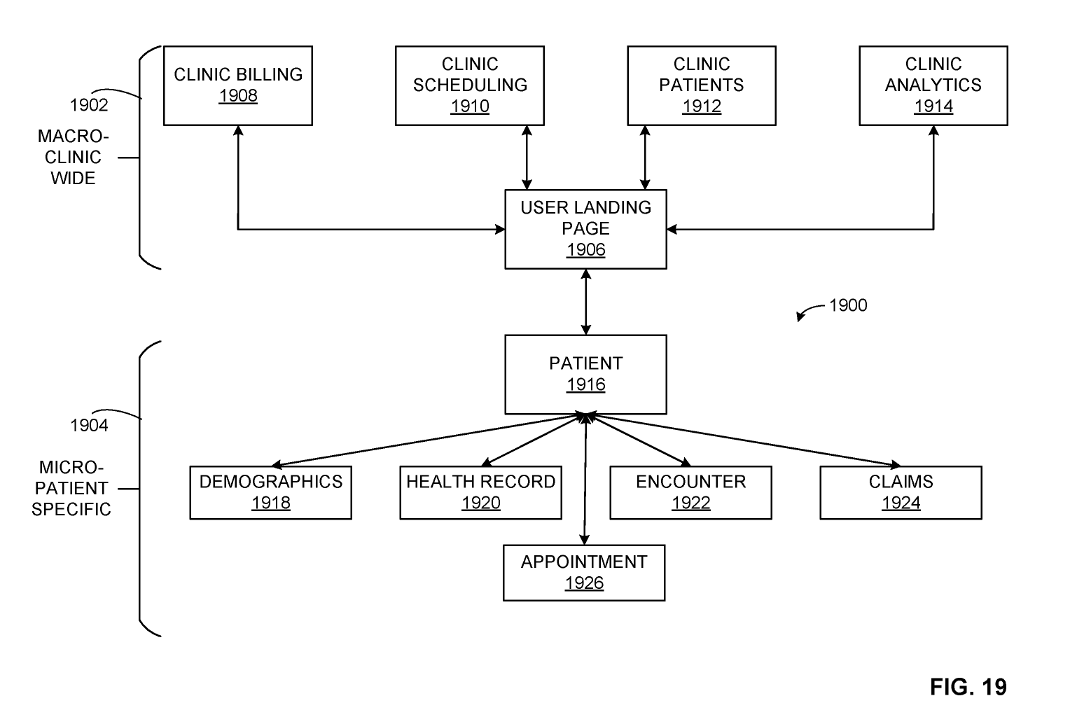

FIG. 19 illustrates an example user interface framework 1900 including a macro (e.g., clinic-wide) interface view 1902 and a micro (e.g., patient-specific) interface view 1904. As shown in the example of FIG. 19, a user landing web page 1906 can serve as a base or starting point for the user's navigation of a graphical user interface generated by the interface engine 1650, for example. From the landing page 1906, the user (e.g., the patient, the provider, etc.) can access information from the macro view 1902 and/or from the micro view 1904. By toggling between the macro 1902 and micro 1904 views, the user can work with information at both an organizational or group level and at a patient level. Based on role (e.g., patient, provider, etc.) different information can be surfaced in each view 1902, 1904 depending upon permission/access restriction associated with the role and/or particular user, for example.

As shown in the example of FIG. 19, information can be rendered via the user landing page 1906 in the macro view 1902 across clinic billing 1908, across clinic scheduling 1910, across clinic patients 1912, and/or across clinical analytics 1914, for example. Thus, the user can access patient and/or other information for one or more patients 1912 across multiple billings 1908 for a clinic and/or other healthcare facility, as well as across multiple schedules 1910 for the clinic/facility. Analytics 1912 can also be leveraged for one or more patients via the landing page 1906 interface, for example.

Switching to the micro view 1904, information can be rendered via the user landing page 1906 to focus on a particular patient 1916 (e.g., the user, the user's patient, the user's child, etc.). For the particular patient 1916, demographic information 1918, patient health record 1920 (e.g., EMR, EHR, etc.), encounter information 1922, claims 1924, appointment/scheduling information 1926, etc., can be visualized via the landing page interface 1906. Such patient 1916 information can include one or more tasks to be understood, worked, reviewed/approved, etc., via the landing page interface 1906, for example.

For example, based on role/persona (e.g., patient, biller, provider, front desk, nursing staff, etc.), different macro 1902 and micro 1904 options can be configured for the landing page 1906. For example, for a biller persona, the landing page 1906 can be configured in the macro view 1902 for a billing dashboard 1908 across the clinic. In the micro view 1904, the biller persona can be configured to view claims 1924, chart 1920, appointments 1926, and profile information 1918, for example. For a provider persona, the macro view 1902 can be arranged to provide schedule 1910 information/interaction via the landing page 1906, and the micro view 1904 can be arranged to provide chart 1924, profile 1918, and appointment 1926 information, for example. For a front desk persona, the macro view 1902 includes cross-clinic(s) scheduling 910, while the micro view 1904 switches to a view of appointment(s) 1926 and profile 1918 information for the patient 1916, for example. For a nursing staff persona, the macro view 1902 includes the schedule 1910, while the micro view 1904 includes claims 1924, chart 1920, appointment(s) 1926, and profile 1918 information, for example. Thus a workflow associated with the role/persona (e.g., a patient workflow, a provider workflow, a biller workflow, a front desk workflow, a nurse workflow, etc.) can be facilitated via the interface and its landing page 1906 enabling a switch between macro 1902 and micro 1904 views.

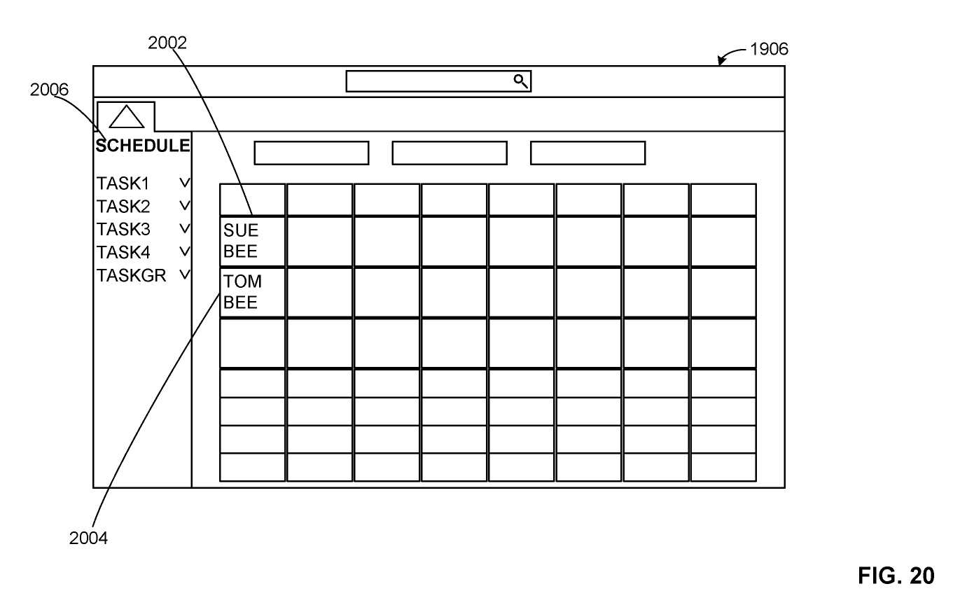

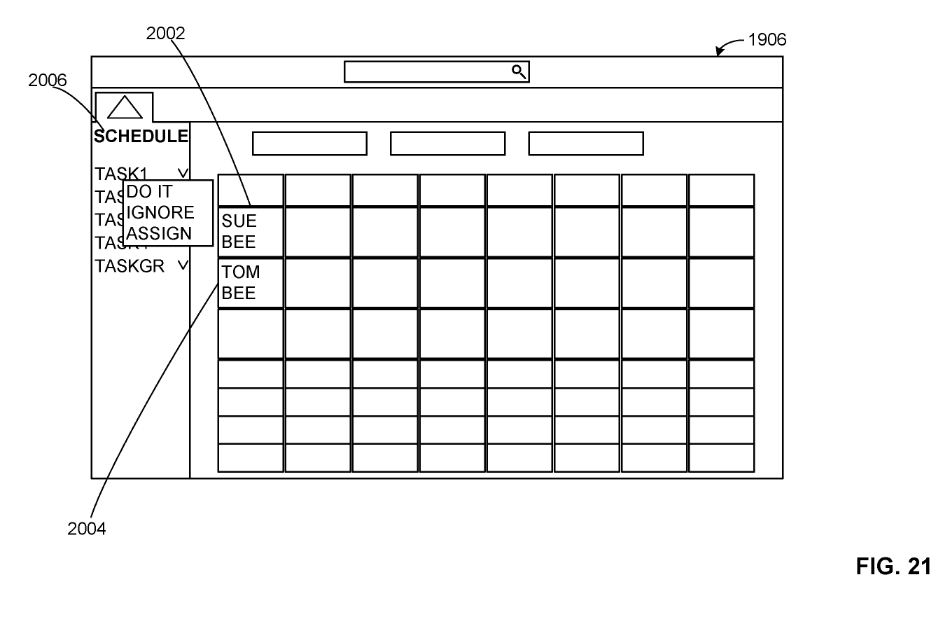

For example, the landing page 1906 can be a curated landing page 1906 such as illustrated in the example of FIG. 20. The example landing page 1906 includes a list of patients 2002, 2004, and a schedule of tasks 2006. As shown in the example of FIG. 21, for each task 2006, a pop-up menu 2102 appears based on selection of the task 2006 For example, selecting Task 1 generates the pop-up menu 2102 including options to do the task, ignore the task, or assign the task to someone, etc. In certain examples, a plurality of task finish types can be provided such as a simple change command, a medium change wizard, a large change navigation in context, etc. Patients 2002, 2004, tasks 2006, options 2102 can vary based on user, role/persona, etc.

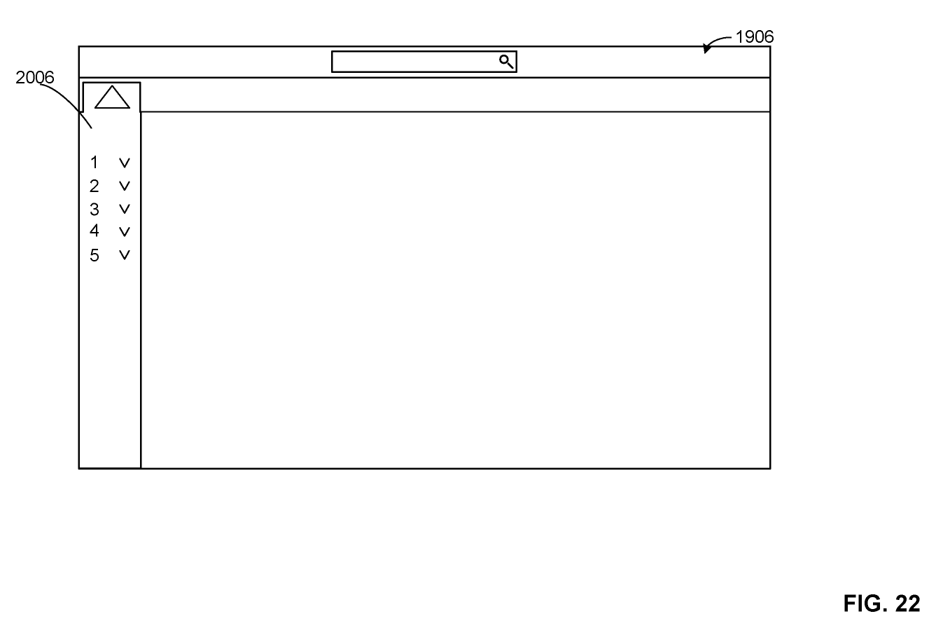

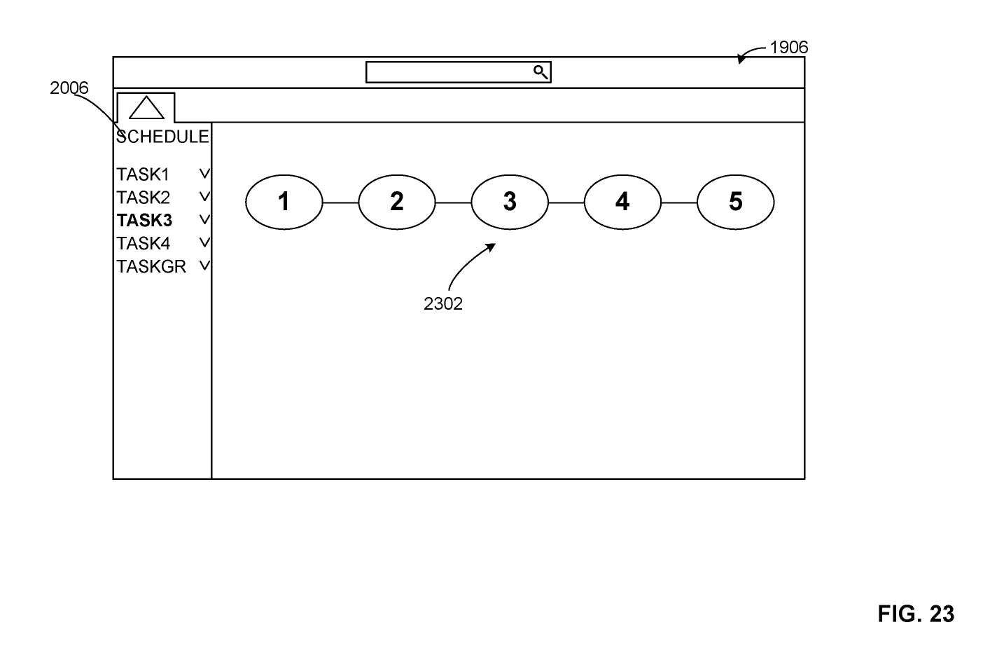

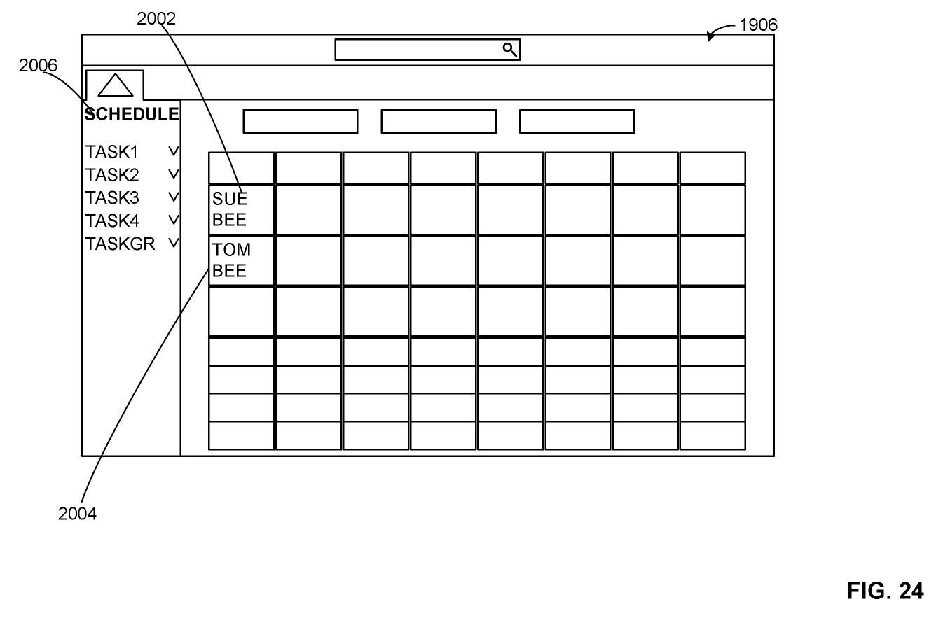

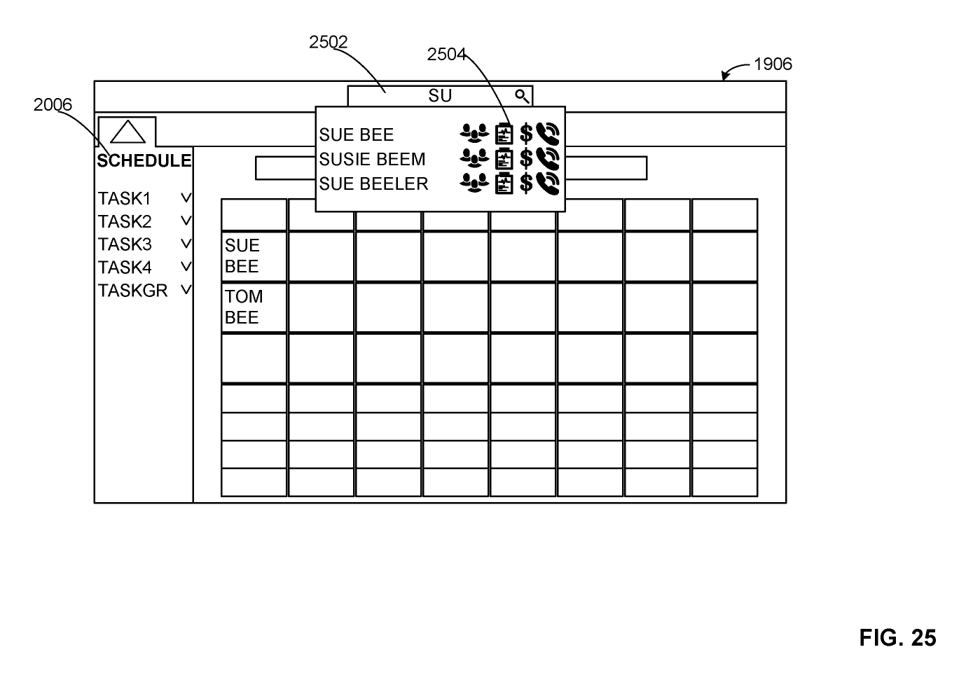

FIG. 22 illustrates an example configuration of the interface 1906 in which a task 2006 is navigated in context. FIG. 23 illustrates an example configuration of the interface 1906 showing a wizard indicating selectable options 2302 with respect to one or more tasks 2006. FIG. 24 illustrates an example interface 1906 configuration in which patient(s) 2002, 2004 can be accessed via the schedule of tasks 2006. In FIG. 25, the patient 2002 can be accessed via a search box 2502. Depending upon user access/permission, short cut icons 2504 provide a jump to a specific area such as health record 1920, claims 1924, schedule encounter 1922, etc.

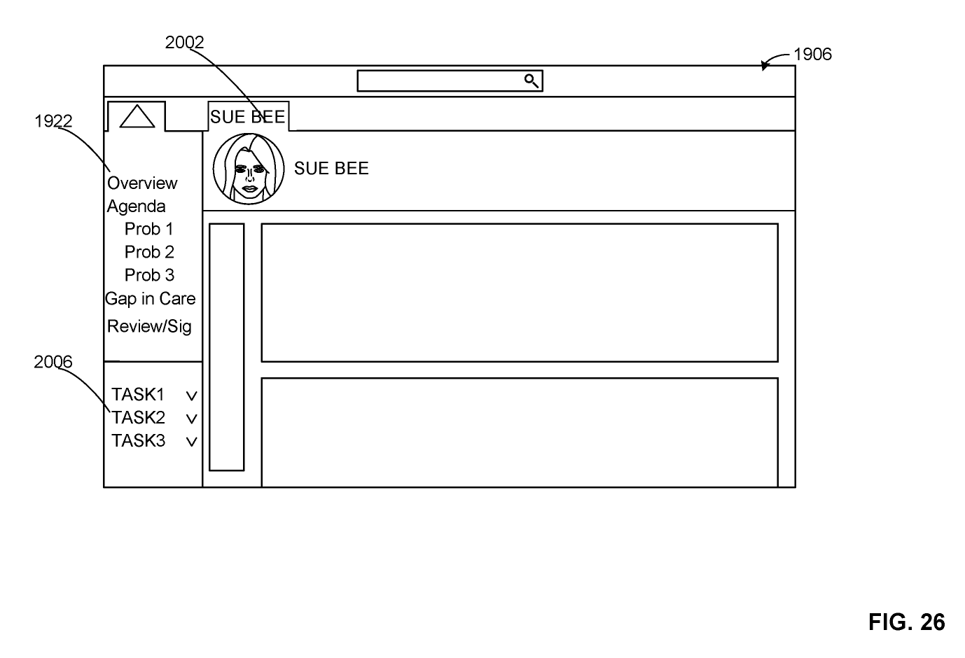

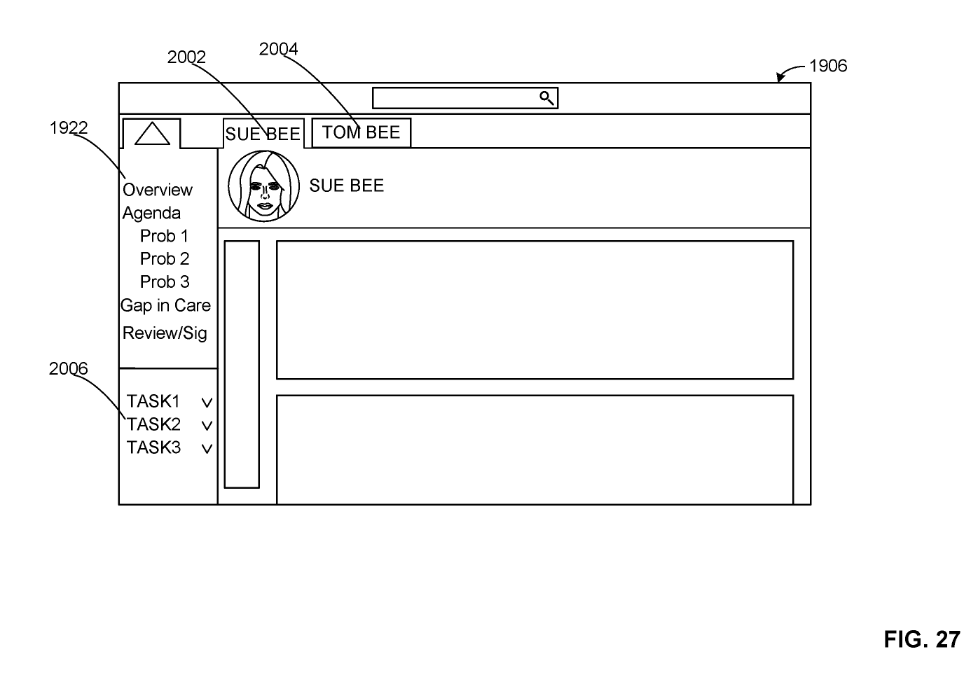

FIG. 26 illustrates an example encounter workflow interface 1906 for a patient 2002. As shown in the example of FIG. 26, an encounter schedule 1922 is provided as well as a list of tasks 2006 associated with the patient encounter. FIG. 27 illustrates a similar encounter interface 1906 except as a family encounter interface with two patients 2002, 2004, rather than one patient 2002.

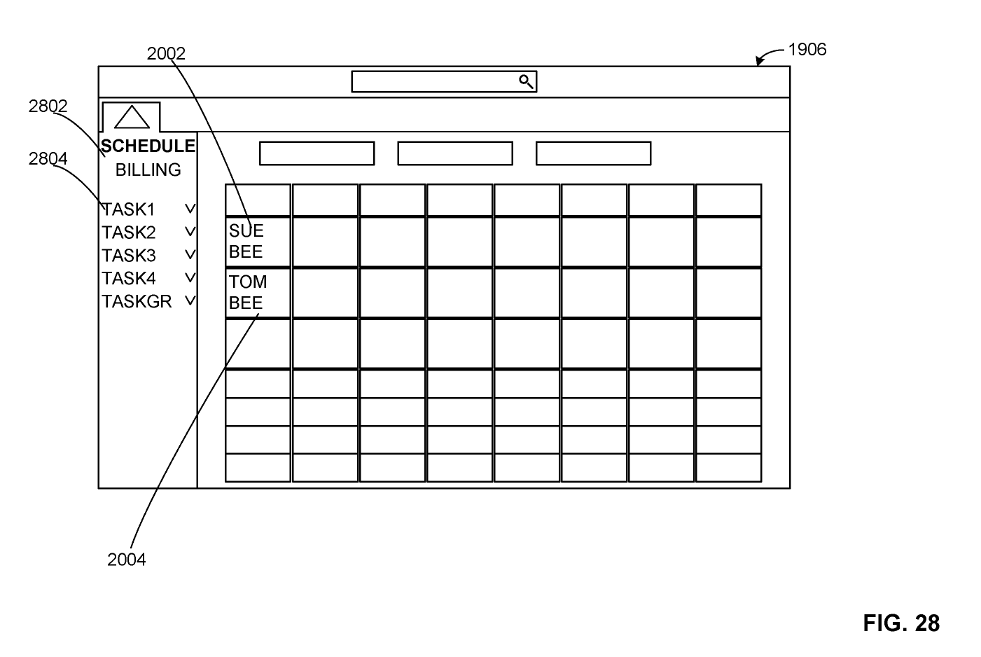

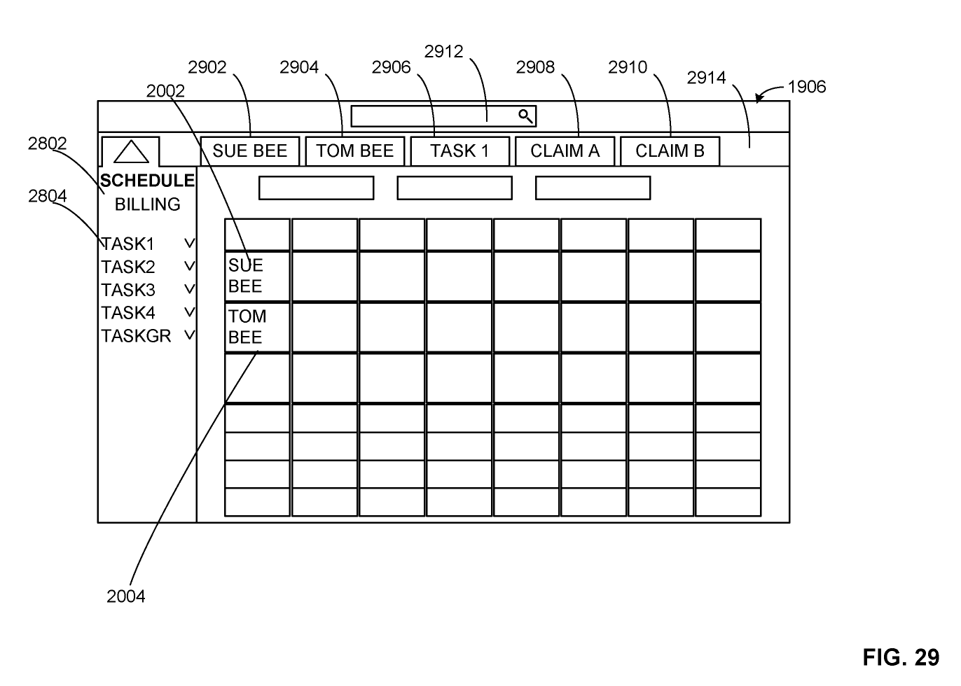

FIG. 28 illustrates an example provider interface 1906 with billing credentials illustrating an example billing schedule 2802 and associated billing tasks 2804 for one or more patients 2002, 2004. In FIG. 29, a multi-tasking view 1906 is shown with a plurality of tabs 2902-2910 indicating an associated view/task. For example, a search 2912 spawns a tab 2902-2910 in the interface 1906. A user can also spawn a tab 2902-2910 by selecting a tab bar 2914. The user can spawn a window by double-clicking and/or otherwise selecting the tab bar 2914, for example.

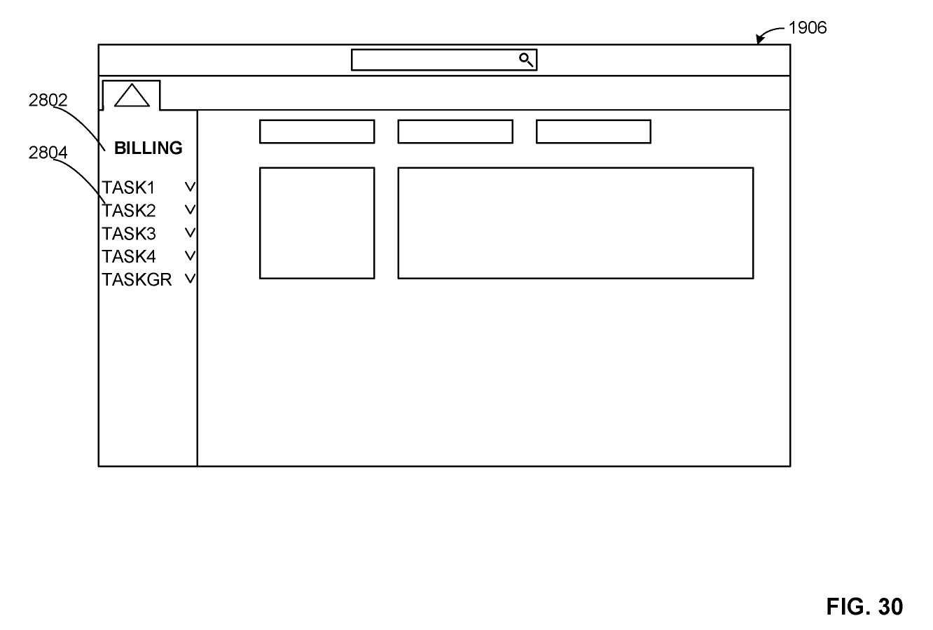

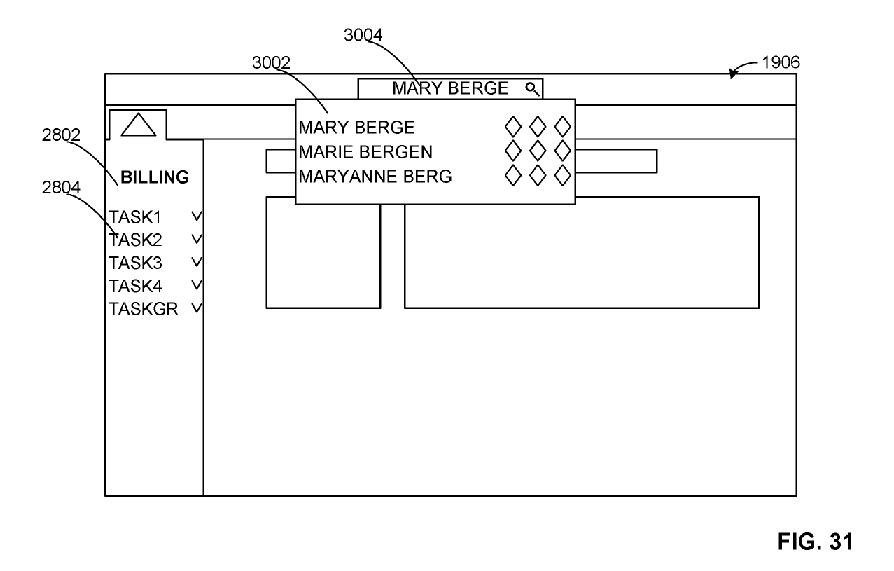

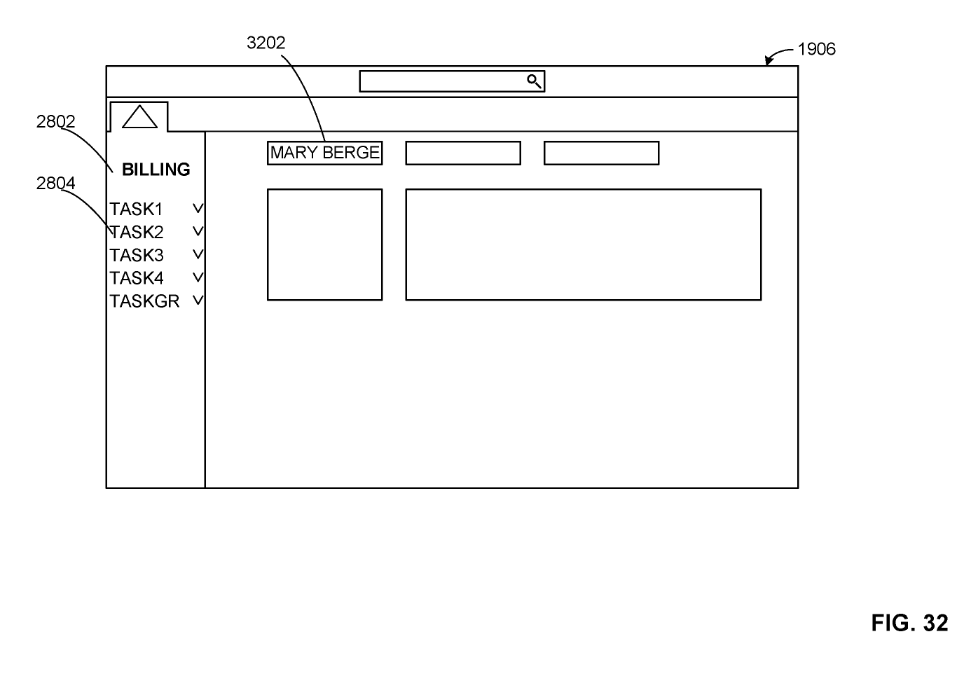

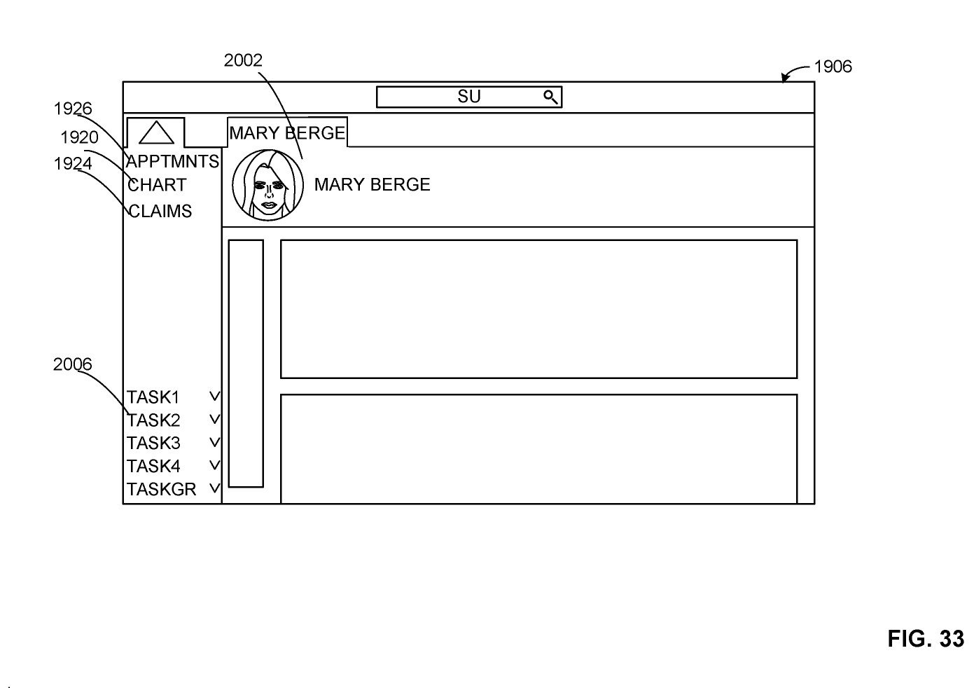

FIG. 30 shows another example implementation of the curated landing page 1906. In the example of FIG. 30, billing 2802 and associated tasks 2804 are provided for user selection. In the example of FIG. 31, a patient 3002 is accessed via a search 3004 from the landing page interface 1906. In the example of FIG. 32, a patent can be accessed via a drill down or pull down menu 3202. In the example of FIG. 33, a patient module 1916 is provided via the landing page 1906. For a patient 2002, appointment(s) 1926, chart 1920, claims(s) 1924, etc., can be presented, as well as tasks 2006 to be executed via the interface 1906, for example.

Thus, certain examples facilitate virtual patient visits, real time scheduling, payment options, and patient enablement through meaningful display, access to medical expertise/services, and educational materials provided by the curated landing page 1906. Certain examples drive patient scenario maps including search/discovery for information regarding a condition/symptom/complaint, registration for further information/assessment/treatment, provider selection/scheduling, pre-visit gathering of information/reason for visit, check-in, encounter/assessment between patient and healthcare provider, check-out, and post-visit instructions/follow-up, etc. Clearly, manual human action is no substitute for such interfaces accessible via the landing page 1906 in macro 1902 and/or micro 1904 views. The role-based navigation and switchable macro 1902/micro 1904 views represent a technological improvement in webpage and/or other interface technology and provide benefits to computer operation, interface navigation, and user workflow through the macro 1902/micro 1904 dynamic and selective, role and view-based surfacing of content and functionality provided via the interface 1906.

Engaging ambulatory healthcare providers can be a cumbersome process for patients that can lead to lack of adoption of preventative or chronic care by patients. By focusing on the patient user when building practice management capabilities within the system 1600, 1900, the interface 1906 can facilitate increased interaction with ambulatory practices as a patient's first place to engage in his or her healthcare, for example. In addition, such a system 1600 and interface 1906 can help increase adherence to care plans. Certain examples focus on system 1600 and interface 1906 foundations for a patient as a user who will access views of the practice schedule, registration, pre-appointment tasks, post-appointment tasks, and financial responsibility options.

In certain examples, infrastructure such as the system 1600 and interface 1906 facilitate mobile access to solutions that streamline user workflows with exception-based tasking (e.g., tasking 2006) for healthcare practices and patients to engage in healthcare. Allowing practices and patients to manage appointment scheduling, pre-appointment work items, financial responsibility, coordinating gaps in care adherence, automating claims, and payment, additional EDI transactions, etc., provides improved reliability, access, and robustness of healthcare computing systems.

Thus, using a role-based GUI display, a user can switch between macro 1902 and micro 1904 views and surface information based on triggers to de-clutter the interface 1906. The smart rule-based engine 640, 1630 drives the interface 1906 and can also auto-curate the content and/or functionality displayed. Based on login and/or other identification/authorization information, the rules engine 640, 1630 can generate personalized recommendations for a particular patient including tasks to do, activities in which to participate, etc.

Users, such as patients, providers, etc., can easily navigate through coordination of care with an ability to schedule appointments, understand financial responsibility with eligibility and patient liability estimation, manage registration updates online, identify open treatment or health plans and take action, etc. Certain examples remedy the problem of lack of patient engagement in the healthcare system due to a cumbersome manual process, lack of synchronous communication between care providers and patients by facilitating an overall understanding of the impact of not actively communicating with the patient's clinical support team care, as well as simplifying the financial process to enable patients to focus on their care rather than their bills.

Certain examples provide infrastructure support for a customizable interface 1906 providing an improved user experience through role-based, global navigation via the GUI. Based on role, the landing page 1906 can serve up page(s) and task(s) according to the role, for example. Based on how the user logs in, a particular view is provided via the landing page 1906, for example. By allowing data to persist and/or be carried over when the user toggles between views 1902, 1904, users are saved repeated entry of data and correlations can be established between the views 1902, 1904, for example.

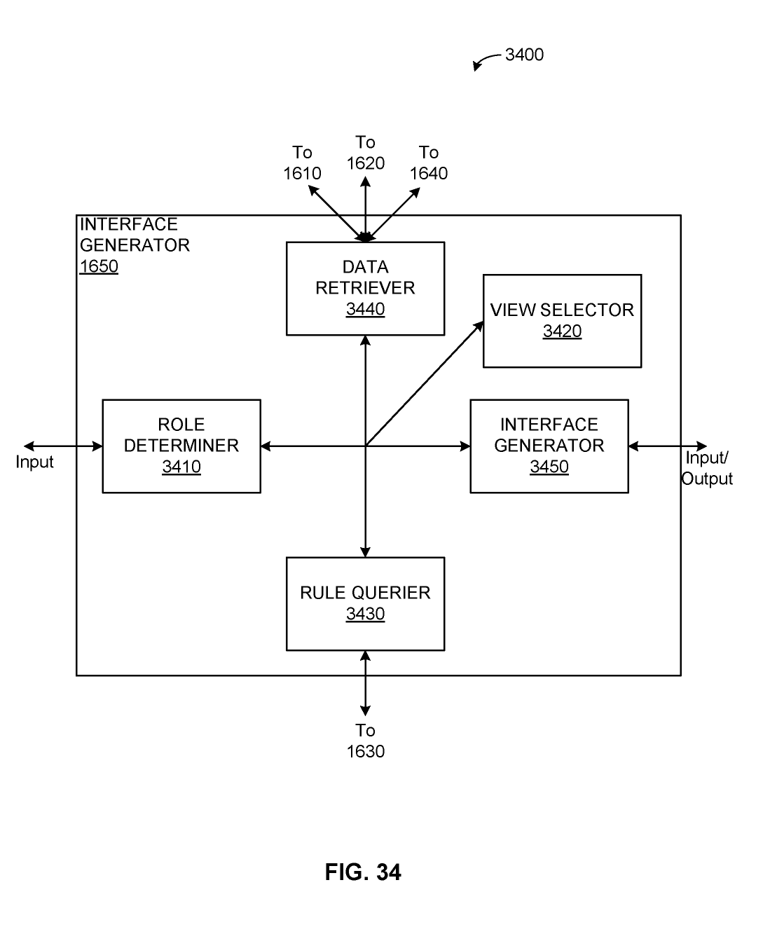

FIG. 34 illustrates an example implementation of the interface engine 1650 of FIG. 16. The example interface engine 1650 includes a role determiner 3410, a view selector 3420, a rule querier 3430, a data retriever 3440, and an interface generator 3450. As shown in the example of FIG. 34, the role determiner 3410 receives an input, such as a user identification (e.g., username, etc.), authentication (e.g., token, key, etc.), etc., to identify the user. The input is used by the role determiner 3410 to identify the user and a role and/or other persona associated with the user. The role determiner 3410 provides role and/or other user identification to the view selector 3420, the rule querier 3430, the data retriever 3440, and/or the interface generator 3450.

The example view selector 3420 determines a view selected for display via the interface (e.g., the landing page 1906, etc.). For example, the macro 1902 or micro 1904 can be specified as a default page, selected by a user, specified by a program/parameter, etc. The view selector 3420 helps to determine which rule(s) are queried by the rule querier 3430 and/or which data is retrieved from one or more additional systems by the data retriever 3440, for example.

The example rule querier 3430 queries the rules engine 1630, 640 for content, view information, data retrieval parameters, interface configuration information, etc., based on the role and/or view input. For example, the rule querier 3430 queries the rules engine 1630 to retrieve rule(s) to auto-curate the content and/or functionality displayed based on user, selected view, etc. The rule querier 3430 can retrieve trigger(s) from the rules engine 1630 to surface particular content and/or functionality and hide other content/functionality based on the role, view, etc. Such information can be provided to the data retriever 3440 to retrieve relevant data from the workflow processor 1610, phase monitor 1620, EMR 1640, etc. The information can also be provided to the interface generator 3450 to render the GUI for the user, for example.

The example data retriever 3440 takes role, view, and/or rule information and retrieves information from one or more connected components such as the workflow processor 1610, phase monitor 1620, EMR 1640, etc. For example, the data retriever 3440 can retrieve content from the EMR 1640 according to the rule(s) from the rule querier 3430 and based on the role and/or view provided by the role determiner 3410 and/or view selector 3420, respectively. The data retriever 3440 can retrieve information from the workflow processor 1610 and/or phase monitor 1620 to provide functionality based on workflow phase, task(s), etc. The data retriever 3440 provides retrieved information to the interface generator 3450.

The example interface generator 3450 curates the interface 1906 based on the role, view, and information retrieved, as specified by the rule(s). The interface generator 3450 provides particular content, functionality, etc., in the macro view 1902 and/or the micro view 1904 and facilitates toggling between the views 1902, 1904 and data sharing between the views 1902, 1904, for example. The interface generator 3450 leverages the rules engine 1630 (via the rule querier 3430) and information provided by the role determiner 3410, view selector 3420, and data retriever 3440 to generate a dynamic, role- and view-based interface accessible via the landing page 1906 to drive customized healthcare workflows for patient, practitioner, etc.

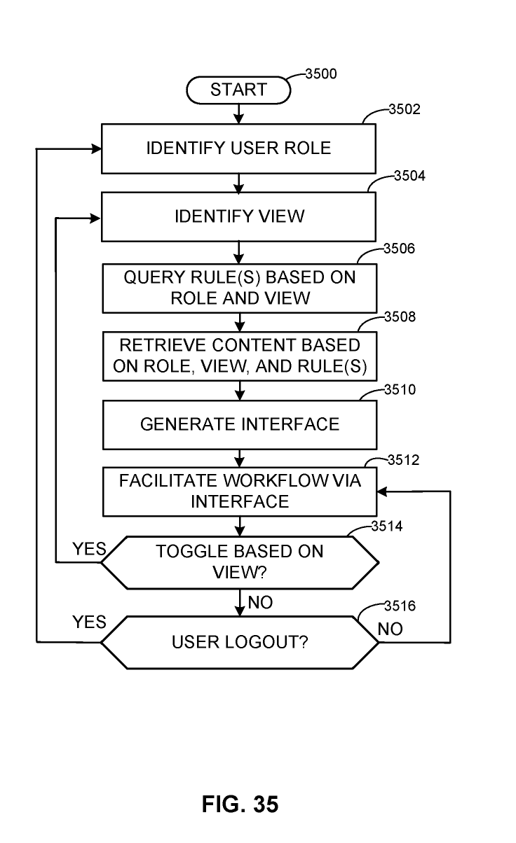

FIG. 35 illustrates a flow diagram of an example method 3500 of user interface generation and healthcare workflow management using the example workflow processor 1610, phase monitor 1620, rules engine 1630, EMR 1640, and interface generator 1650. At block 3502, a user role is identified. For example, the role determiner 3410 receives an input, such as a user identification (e.g., username, etc.), authentication (e.g., token, key, etc.), etc., to identify the user. The input is used by the role determiner 3410 to identify the user and a role and/or other persona associated with the user.

At block 3504, an interface view is identified. For example, the example view selector 3420 determines a view selected for display via the interface (e.g., the landing page 1906, etc.). For example, the macro 1902 or micro 1904 can be specified as a default page, selected by a user, specified by a program/parameter, etc.

At block 3506, rule(s) are queried based on the identified role and view. For example, the example rule querier 3430 queries the rules engine 1630, 640 for content, view information, data retrieval parameters, interface configuration information, etc., based on the role and/or view input. For example, the rule querier 3430 queries the rules engine 1630 to retrieve rule(s) to auto-curate the content and/or functionality displayed based on user, selected view, etc. The rule querier 3430 can retrieve trigger(s) from the rules engine 1630 to surface particular content and/or functionality and hide other content/functionality based on the role, view, etc.

At block 3508, content is retrieved based on the role, view, and rule(s). For example, the example data retriever 3440 takes role, view, and/or rule information and retrieves information from one or more connected components such as the workflow processor 1610, phase monitor 1620, EMR 1640, etc. For example, the data retriever 3440 can retrieve content from the EMR 1640 according to the rule(s) from the rule querier 3430 and based on the role and/or view provided by the role determiner 3410 and/or view selector 3420, respectively. The data retriever 3440 can retrieve information from the workflow processor 1610 and/or phase monitor 1620 to provide functionality based on workflow phase, task(s), etc.

At block 3510, the interface is generated. For example, the example interface generator 3450 curates the interface 1906 based on the role, view, and information retrieved, as specified by the rule(s). The interface generator 3450 provides particular content, functionality, etc., in the macro view 1902 and/or the micro view 1904 and facilitates toggling between the views 1902, 1904 and data sharing between the views 1902, 1904, for example.

At block 3512, a healthcare workflow (e.g., patient, provider, etc.) is facilitated. For example, the interface generator 3450 leverages the rules engine 1630 (via the rule querier 3430) and information provided by the role determiner 3410, view selector 3420, and data retriever 3440 to generate a dynamic, role- and view-based interface accessible via the landing page 1906 to drive customized healthcare workflows for patient, practitioner, etc. A patient can log in to a micro view 1904 of the interface 1906 to view his or her health information, identify a practitioner, register for an appointment, complete pre-visit documentation, execute a virtual visit, check in, document the visit, check-out, and conduct post-visit follow-up and bill payment via the interface 1906, for example. A clinician can log in to a macro 1902 and/or micro 1904 view of the interface 1906 to retrieve patient records, view his/her schedule, prepare for a patient visit, order labs/exams, conduct the patient encounter, document analysis and/or other results, generate orders (e.g., prescription, lab, therapy, device, etc.), and discharge the patient with instructions via the interface 1906, for example.

At block 3514, a toggle between views 1902, 1904 is detected via the interface 1906. If a toggle is detected, then the view 1902, 1904 switches and control reverts to block 3504 to identify the view 1902, 1904. Content and/or context information can be transferred from one view 1902, 1904 to the other view 1902, 1904 based on user, rule, etc.

At block 3516, if a user logout is detected, then control reverts to block 3502 to identify a role of a logging in user. If no user logout is detected, then control reverts to block 3512 to facilitate a user workflow via the interface 1906.

One skilled in the art will appreciate that embodiments of the invention may be interfaced to and controlled by a computer readable storage medium having stored thereon a computer program. The computer readable storage medium includes a plurality of components such as one or more of electronic components, hardware components, and/or computer software components. These components may include one or more computer readable storage media that generally stores instructions such as software, firmware and/or assembly language for performing one or more portions of one or more implementations or embodiments of a sequence. These computer readable storage media are generally non-transitory and/or tangible. Examples of such a computer readable storage medium include a recordable data storage medium of a computer and/or storage device. The computer readable storage media may employ, for example, one or more of a magnetic, electrical, optical, biological, and/or atomic data storage medium. Further, such media may take the form of, for example, floppy disks, magnetic tapes, CD-ROMs, DVD-ROMs, hard disk drives, and/or electronic memory. Other forms of non-transitory and/or tangible computer readable storage media not list may be employed with embodiments of the invention.

A number of such components can be combined or divided in an implementation of a system. Further, such components may include a set and/or series of computer instructions written in or implemented with any of a number of programming languages, as will be appreciated by those skilled in the art. In addition, other forms of computer readable media such as a carrier wave may be employed to embody a computer data signal representing a sequence of instructions that when executed by one or more computers causes the one or more computers to perform one or more portions of one or more implementations or embodiments of a sequence.

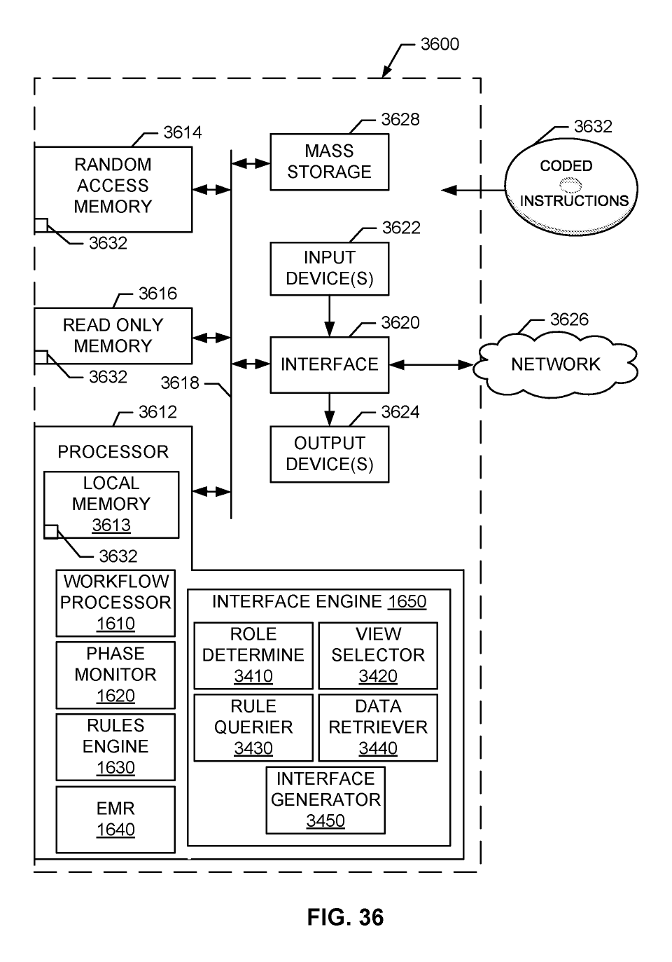

FIG. 36 is a block diagram of an example processor platform 3600 capable of executing instructions to implement the example systems and methods of FIGS. 1-35. The processor platform 3600 can be, for example, a server, a personal computer, a mobile device (e.g., a cell phone, a smart phone, a tablet such as an IPAD™), a personal digital assistant (PDA), an Internet appliance, or any other type of computing device.

The processor platform 3600 of the illustrated example includes a processor 3612. Processor 3612 of the illustrated example is hardware. For example, processor 3612 can be implemented by one or more integrated circuits, logic circuits, microprocessors or controllers from any desired family or manufacturer.

Processor 3612 of the illustrated example includes a local memory 3613 (e.g., a cache). Processor 3612 of the illustrated example is in communication with a main memory including a volatile memory 3614 and a non-volatile memory 3616 via a bus 3618. Volatile memory 3614 can be implemented by Synchronous Dynamic Random Access Memory (SDRAM), Dynamic Random Access Memory (DRAM), RAMBUS Dynamic Random Access Memory (RDRAM) and/or any other type of random access memory device. The non-volatile memory 3616 can be implemented by flash memory and/or any other desired type of memory device. Access to main memory 3614, 3616 is controlled by a memory controller. The processor 3612, alone or in conjunction with the memory 3613, can be used to implement the workflow processor 1610, the phase monitor 1620, the rules engine 1630, the EMR 1640, the interface engine 1650 and/or other part of the systems disclosed herein. For example, the processor 3612 can be used to implement the example role determiner 3410, view selector 3420, rule querier 3430, data retriever 3440, and interface generator 3450 of the example interface engine 1650.

Processor platform 3600 of the illustrated example also includes an interface circuit 3620. Interface circuit 3620 can be implemented by any type of interface standard, such as an Ethernet interface, a universal serial bus (USB), and/or a PCI express interface.

In the illustrated example, one or more input devices 3622 are connected to the interface circuit 3620. Input device(s) 3622 permit(s) a user to enter data and commands into processor 3612. The input device(s) 3622 can be implemented by, for example, an audio sensor, a microphone, a camera (still or video), a keyboard, a button, a mouse, a touchscreen, a track-pad, a trackball, isopoint and/or a voice recognition system.

One or more output devices 3624 are also connected to interface circuit 3620 of the illustrated example. Output devices 3624 can be implemented, for example, by display devices (e.g., a light emitting diode (LED), an organic light emitting diode (OLED), a liquid crystal display, a cathode ray tube display (CRT), a touchscreen, a tactile output device, a light emitting diode (LED), a printer and/or speakers). Interface circuit 3620 of the illustrated example, thus, typically includes a graphics driver card, a graphics driver chip or a graphics driver processor.

Interface circuit 3620 of the illustrated example also includes a communication device such as a transmitter, a receiver, a transceiver, a modem and/or network interface card to facilitate exchange of data with external machines (e.g., computing devices of any kind) via a network 3626 (e.g., an Ethernet connection, a digital subscriber line (DSL), a telephone line, coaxial cable, a cellular telephone system, etc.).

Processor platform 3600 of the illustrated example also includes one or more mass storage devices 3628 for storing software and/or data. Examples of such mass storage devices 3628 include floppy disk drives, hard drive disks, compact disk drives, Blu-ray disk drives, RAID systems, and digital versatile disk (DVD) drives.

Coded instructions 3632 associated with any of FIGS. 1-35 can be stored in mass storage device 3628, in volatile memory 3614, in the non-volatile memory 3616, and/or on a removable tangible computer readable storage medium such as a CD or DVD.

It may be noted that operations performed by the processor platform 3600 (e.g., operations corresponding to process flows or methods discussed herein, or aspects thereof) may be sufficiently complex that the operations may not be performed by a human being within a reasonable time period.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

1. An apparatus comprising:
a rules engine including rules for workflow execution and interface generation; and
an interface engine to generate a graphical user interface based on a role of a user and one or more rules, the interface engine to at least:
query the rules engine to provide the one or more rules based on the role;
retrieve content based on the role and the one or more rules;
dynamically generate the graphical user interface showing a first view based on the retrieved content arranged according to the role and the one or more rules;
facilitate execution of a healthcare workflow by the user via the graphical user interface; and
toggle between the first view and a second view, the second view dynamically generated based on the retrieved content arranged according to the role and the one or more rules.

2. The apparatus of claim 1, wherein the first view includes at least one of a macro view across a healthcare facility and a micro view of a particular patient, and wherein the second view includes at least one of the macro view and the micro view.

3. The apparatus of claim 1, wherein the interface engine maintains content and context in toggling between the first view and the second view.

4. The apparatus of claim 1, wherein the user includes a patient.

5. The apparatus of claim 4, wherein the first view includes a micro view of the patient and wherein the second view includes a macro view associated with a provider, the user restricted to a subset of the macro view related to the user.

6. The apparatus of claim 1, wherein the one or more rules includes a rule correlating display of at least one of the first view or the second view to a phase of the healthcare workflow.

7. The apparatus of claim 1, wherein the role includes at least one of patient, biller, provider, front desk, or nursing staff.

8. A computer-implemented method comprising:
identifying, by executing an instruction using a processor, a role associated with a user;
querying, by executing an instruction using the processor, a rules engine to provide one or more rules based on the role;
retrieving, by executing an instruction using the processor, content based on the role and the one or more rules;
dynamically generating, by executing an instruction using the processor, a graphical user interface showing a first view based on the retrieved content arranged according to the role and the one or more rules;
facilitating, by executing an instruction using the processor, execution of a healthcare workflow by the user via the graphical user interface; and
toggling, by executing an instruction using the processor, between the first view and a second view, the second view dynamically generated based on the retrieved content arranged according to the role and the one or more rules.

9. The method of claim 8, wherein the first view includes at least one of a macro view across a healthcare facility and a micro view of a particular patient, and wherein the second view includes at least one of the macro view and the micro view.

10. The method of claim 9, wherein content and context are maintained in toggling between the first view and the second view.

11. The method of claim 8, wherein querying further includes querying the rules engine to provide one or more rules based on the role and the first view.

12. The method of claim 1, wherein the user is a patient, wherein the first view includes a micro view of the patient, and wherein the second view includes a macro view associated with a provider, the user restricted to a subset of the macro view related to the user.

13. The method of claim 8, wherein the one or more rules includes a rule correlating display of at least one of the first view or the second view to a phase of the healthcare workflow.

14. The method of claim 8, wherein the role includes at least one of patient, biller, provider, front desk, or nursing staff.

15. A tangible computer-readable storage medium including instructions which, when executed, particularly configure a processor to at least:
identify a role associated with a user;
query a rules engine to provide one or more rules based on the role;
retrieve content based on the role and the one or more rules;
dynamically generate a graphical user interface showing a first view based on the retrieved content arranged according to the role and the one or more rules;
facilitate execution of a healthcare workflow by the user via the graphical user interface; and
toggle between the first view and a second view, the second view dynamically generated based on the retrieved content arranged according to the role and the one or more rules.

16. The storage medium of claim 15, wherein the first view includes at least one of a macro view across a healthcare facility and a micro view of a particular patient, and wherein the second view includes at least one of the macro view and the micro view.

17. The storage medium of claim 15, wherein content and context are maintained in toggling between the first view and the second view.

18. The storage medium of claim 15, wherein the processor is to query the rules engine to provide one or more rules based on the role and the first view.

19. The storage medium of claim 15, wherein the one or more rules includes a rule correlating display of at least one of the first view or the second view to a phase of the healthcare workflow.

20. The storage medium of claim 15, wherein the role includes at least one of patient, biller, provider, front desk, or nursing staff.
